(19) United States
(12) Patent Application Publication
(10) Pub. No.: US 2025/0009940 A1
(43) Pub. Date: Jan. 9, 2025

(54) ANTIMICROBIAL WRAPS FOR MEDICAL IMPLANTS

(71) Applicant: BOARD OF REGENTS, THE UNIVERSITY OF TEXAS SYSTEM, Austin, TX (US)

(72) Inventors: Joel ROSENBLATT, Houston, TX (US); Issam RAAD, Houston, TX (US)

(73) Assignee: BOARD OF REGENTS, THE UNIVERSITY OF TEXAS SYSTEM, Austin, TX (US)

(21) Appl. No.: 18/708,282

(22) PCT Filed: Nov. 17, 2022
(86) PCT No.: PCT/US2022/080031

§ 371 Date: May 8, 2024

Related U.S. Application Data
(60) Provisional application No. 63/280,945, filed on Nov. 18, 2021.

(51) Int. Cl. A61L 27/50; A61L 27/58

(52) U.S. Cl. CPC A61L 27/502; A61L 27/58; A61L 2300/404; A61L 2300/41

(57) ABSTRACT

Biodegradable antimicrobial films with improved surface and handling properties are provided that are solid at room temperature and substantially liquefy in situ after implantation into a mammal, such as a human patient. Methods of using the films to cover a medical device, such as a breast implant, prior to insertion into a subject are also provided. The biodegradable films may contain a drug to reduce inflammation or capsular contracture. Methods of making biodegradable films are also provided.

This application claims the benefit of U.S. Provisional Patent Application No. 63/280,945, filed Nov. 18, 2021, the entirety of which is incorporated herein by reference.

### BACKGROUND OF THE INVENTION

### 1. Field of the Invention

The present invention relates generally to the field of medicine. More particularly, it concerns antimicrobial films and coverings for medical devices, and related methods.

### 2. Description of Related Art

Breast reconstruction is frequently performed following mastectomies. Breast implants or tissue expanders are frequently used. Infection is a significant problem associated with breast implants and tissue expanders for recovering cancer patients. Infection rates for reconstruction cases have been estimated to range from 2-24% (Pittet el al., 2005). Other than the direct systemic complications of infection, local complications can cause discomfort, cosmesis, capsule formation and hardening and can lead to implant removal or replacement. Current protocol is to bathe breast implant and tissue expander devices in an aqueous solution of three different antibiotics for 5-15 minutes prior to insertion. Most implants are made from silicone rubber which is highly hydrophobic so the antibiotic solution rolls off the device after it is removed from the antibiotic bath, hence very little antibiotic is actually carried into the implant tissue pocket following insertion. In a retrospective study of breast implant infections following reconstructive surgery, 79% of cases had appropriate antibiotic irrigation performed prior to placement but 63% had breakthrough infections despite that (Viola et al., 2014).

Following insertion, a drainage catheter is usually left in place for a week or so which can be a conduit for bacterial access to the device. Furthermore, although the skin flap is eventually closed, breast tissue has high levels of endogenous bacterial flora that can access and colonize the device. The factors create a prolonged need for infection protection beyond the insertion procedure itself that is not met using the current standard of care. The bathing procedure adds to valuable operating room (OR) time and because of the size of the implant, significant volumes of antibiotic solution are required to bathe the implant. Clearly, there is a need for methods for reducing the risk of infection associated with implanting a medical device or prosthesis, such as a breast implant.

### SUMMARY OF THE INVENTION

The present invention overcomes limitations in the prior art by providing, in certain aspects, biodegradable films with improved surface and handling properties. The biodegradable film may contain highly plasticized gelatin comprising 30-60% plasticizer and an antimicrobial or bioactive agent. The film can be used to partially or completely cover an implant prior to implementation in a mammalian subject.

In some aspects, the instant disclosure provides meltable films with improved surface properties that can melt after implantation. In particular, antimicrobial wraps provided herein may display improved surface properties as compared to meltable wraps formed using highly plasticized gelatin comprising 30-60% plasticizer described in U.S. Pat. No. 10,953,137. In some aspects, it has been observed that freezing antimicrobial highly plasticized gelatin wraps can result in wraps with superior handling properties, such as reduced tackiness for improved handling and covering a medical implant while displaying sufficient strength and flexibility to be able to conformally wrap and adhere to a medical implant in a solid state without additional securement. As shown in the below Examples, highly plasticized gelatin wraps of U.S. Pat. No. 10,953,137 (produced via heating to dissolve the components, casting and curing the wraps, and cooling and drying at room temperature to solidify the wraps) were observed to produce a final wrap with a tacky or gluey surface texture. This physical quality of tackiness was undesirable for several reasons. The tackiness was undesirable in that a release liner was required to prevent the wrap from sticking to its package, and also that some of the antimicrobial agents or medication(s) loaded into the wrap and/or residing at the wrap surface could transfer to the surface of the liner or package which the wrap was contained in and would be lost for therapeutic application. Additionally, tackiness could complicate the manual deployment of the wrap around an implant or surgical site by clinicians, e.g., during a surgical procedure. For example, tackiness of the wrap could be particularly problematic or difficult when deployed using minimally invasive surgical tools such as trocars and laparoscopic forceps.

In some aspects, it has been observed that freezing or freeze-drying antimicrobial wraps containing a highly plasticized gelatin can result in wraps with improved physical properties, including improved surface texture and handling characteristics. In contrast to previous methodologies showing that freezing or freeze drying can be used to create holes in a gelatin film, holes were not observed in the antimicrobial wraps containing a highly plasticized gelatin. In addition to the improved physical characteristics and handling properties, holes were not observed in the antimicrobial wraps containing highly plasticized gelatin after freezing or freeze-drying. It is anticipated that large holes in an antimicrobial wrap could decrease the effectiveness of the wrap, e.g., by increasing the chances that a bacterium could pass through the wrap and/or by decreasing the barrier properties of the wrap. Freezing and freeze-drying gelatin has previously been shown to create porous scaffolds. Kang et al. (1999) observed that freezing a swollen gelatin hydrogel phase-separates the water from the gelatin into ice crystals which upon sublimation formed a porous scaffold with very different bulk solid physical properties than the hydrogel (Kang et al., 1999). Van Vlierberghe et al. (2007) showed that manipulating the freezing temperature gradient and then sublimating could significantly modulate pore geometries through the bulk solid (Van Vlierberghe et al. 2007; Van Vlierberghe et al. 2009).

As shown in the below Examples, the inventors surprisingly found that reducing the temperature of a highly plasticized gelatin wrap (e.g., to −20° C.) and then sublimating did not alter the bulk properties of the wrap (did not induce porosity) but did favorably change the surface properties, significantly reducing tackiness. This reduced transfer of incorporated bioactive agents from the wrap surface to the package surface and also made manual manipulation of the wrap (e.g., without the wrap adhering to itself or its package) easier. While not being bound by any particular theory, the inventors have postulated that subjecting the highly plasticized gelatin wrap to temperatures of −20° C. phase might promote separation and removal of the excess water from the wrap as compared to room temperature (20° C.) drying, based on the idea that reducing the temperature for water-glycerol mixtures can increase the water content in the vapor phase, (Zaoui-Djelloul-Daouadji et al, 2014). Surprisingly and unexpectedly, the bulk properties of the wrap were not affected by the cryogenic processing (i.e., the wrap did not form porous scaffolds) and returned to their previous state when warmed to about 20° C. (room temperature). Without wishing to be bound by any theory, the inventors have postulated this may be a result of the high levels of plasticization which may help the wrap retain a homogenous bulk physical structure. At −20° C. the wraps were surprisingly observed to remain ductile and deformable characteristics and thereby resist fracturing and crazing that could weaken or damage the wrap when subsequently warmed and being manipulated for implantation. Without being bound by any theory, it is anticipated that these properties may result from the high degree of plasticization in the wraps. An additional benefit that resulted from the cryoprocessing is reduced degradation (oxidation, epimerization or other chemical-structural changes) to added or impregnated bioactive agents since lower temperatures can retard oxidation and structural rearrangement chemical reactions. Microbiologic performance at 37° C. for Minocycline+Rifampin loaded wraps was also assessed for the antimicrobial wraps prepared using freezing of freeze-drying and was found to be identical to or indistinguishable from wraps not subjected to cryoprocessing and sublimation and prepared by the room temperature drying process used in U.S. Pat. No. 10,953,137. It is anticipated that a variety of temperatures may be used to achieve one or more of the above benefits. For example, the antimicrobial wrap or biodegradable film containing the highly plasticized gelatin may be cooled to from about 0° C. to about −40° C., from about 0° C. to about −30° C., from about −5° C. to about −30° C., from about −10° C. to about −30° C., from about −15° C. to about −25° C., from about −10° C. to about −20° C., or about 0, −5, −10, −15, −20, −25, −30, −35, −40, −45, −49, or any range derivable therein.

As described in the Examples below, highly plasticized gelatin wraps displayed sufficient strength and flexibility to be able to conformally wrap and adhere to a medical implant in a solid state without additional securement, while plasticized gelatin (30% or less plasticizer) was observed to be too stiff to conformally wrap and adhere to a medical implant in the solid state without additional securement to prevent unwrapping. The antimicrobial highly plasticized gelatin wrap can be partially or completely wrapped around a medical implant prior to insertion into the body. The highly plasticized gelatin wrap can partially or substantially liquefy in situ around the implant following implantation and thereby release impregnated antimicrobial agents to inhibit or prevent colonization or infection near the implant, as well as protect mammalian tissues near or in contact with the implant from trauma or other inflammatory stimuli produced by the implantation procedure or presence of the implant. The wrap or film can be trimmed by a surgeon prior to use, e.g., to fit a particular surgical pocket geometry. For example, in some embodiments the film or wrap is provided as a square or rectangle that can be trimmed, if desired, prior to insertion into a subject.

In some aspects, the meltable wraps provided herein may be impregnated with one or more antimicrobial or bioactive agents dissolved in an aqueous solution containing greater than about 50% water. These approaches are in contrast to approaches described in U.S. Pat. No. 10,953,137 that involve mixing the antimicrobial or bioactive agents at high temperature with the highly plasticized gelatin while casting the wrap. In some embodiments, reduced addition of water in the presence of the antimicrobial or bioactive agents is used to impregnate a wrap so less water would need to be removed post-impregnation or post-incorporation. For example, a highly plasticized blank gelatin wrap (not containing any bioactive or antimicrobial agents) can be produced via casting or laminating molten gelatin solutions, and subsequently the wrap is impregnated with the one or more bioactive agents (e.g., antimicrobial agents, anti-inflammatory agents, etc.) by spraying the wrap with an aqueous solution (containing greater than 50% water) that contains the one or more dissolved bioactive agents, thereby imbibing or impregnating by the wrap with the one or more bioactive agents and swelling the wrap. As described in the below examples, the inventors observed that very high loading of bioactive agents could be achieved with this method, without any need to mix them in at high temperatures in one or more layers of the casting/laminating gelatin solutions (as described in U.S. Pat. No. 10,953,137). In some embodiments, uniform impregnation can be attained by moving the spray nozzle uniformly across the surface of the wrap or by using an array of nozzles arranged to create a uniform spraying pattern. In some embodiments, the wrap is sprayed on both sides (e.g., simultaneously sprayed on both sides, sprayed on one side and subsequently sprayed on the other side) to obtain a substantially uniform impregnation of dissolved bioactive agents through the thickness of the wrap. Water miscible non-aqueous solvents can be included in the impregnating mixture to help solubilize bioactive agents. For example, an aqueous solution containing 5-20% (v/v) ethanol (e.g., 10% (v/v) ethanol and 90% (v/v) water) can be used when impregnating an antimicrobial agent (e.g., Rifampin) into a wrap. Other volatile water-miscible or emulsifiable organic and inorganic liquids can be included in the impregnation fluid to load bioactive agents of limited water solubility. Supercriticial fluids can also be used as a carrier for impregnating bioactive molecules, and the carrier fluid(s) or supercritical fluid can be removed post-impregnation, if desired. The impregnating fluid may contain one or more microemulsion, microsuspension, nanoemulsion, and/or nanosuspension, e.g., which may be convected with the impregnating fluid. Spraying can be performed using a pressurized gas (e.g., nitrogen, carbon dioxide, or other a volatile oxygen-free propellant) to reduce oxidative degradation of bioactive agent(s) and also performing the spray process in an oxygen-free (e.g., nitrogen gas) environment. As shown in the below Examples, an advantage of this impregnation method over the casting method described in U.S. Pat. No. 10,953,137 is that incompatible bioactive agents can be separated spatially in a wrap by impregnating one surface or (length×width) region of a wrap with a first bioactive agent and later impregnating a second bioactive agent (e.g., wherein the second bioactive agent is incompatible with the first bioactive agent or would not be applied using the same impregnation solution) in a overlapping or non-overlapping surface or region of the wrap. This process can be used to avoid the need to make separate wraps and laminate pieces into another wrap as described in U.S. Pat. No. 10,953,137 in order to segregate incompatible bioactive agents. It has been observed that these methods can be used to achieve the advantage of incorporating bioactive agents while reducing by greater than 50% the amount of liquid needed that later would be evaporated or sublimed off or the wrap. Heat and other harsh conditions can be applied to remove excess fluid (water) from a wrap since very few or substantially no bioactive agents would be exposed to the heat or harsh conditions. The subsequent incorporation by impregnation of the bioactive agents would thus require less water (aqueous carrier fluid) removal since significantly less is added to the wrap to incorporate the bioactive agents. Additionally, this method of impregnation of a wrap allows for cold solutions (e.g., about 2-10° C.) to be used for impregnation, which may slow or reduce oxidative and/or other degradation of bioactivity that may result from exposing delicate bioactive agents to higher temperature solutions (such as temperatures of a molten gelatin solution temperatures used in casting wrap layers). The inventors surprisingly found that chilled solutions when sprayed onto blank or unimpregnated wraps were rapidly and fully imbibed by the blank or unimpregnated wraps.

In some aspects the pH of the antimicrobial wrap or biodegradable film can be adjusted, e.g., to a pH of about 6-8. For example, in some embodiments the antimicrobial wrap comprises minocycline (optionally in combination with another antimicrobial agent such as rifampin), wherein the wrap has been adjusted to a pH of 6-8 (e.g., pH 7-7.4, or pH 7). In contrast, intravenous administration of minocycline is typically infused intravascularly at pH 4 and the hydrochloride salt is formulated to create a solution pH of about 4 when dissolved in aqueous infusates, in order to stabilize the minocycline. As shown in the below examples, the pH of antimicrobial wraps was adjusted to pH 6-8 without decreasing the observed antimicrobial properties of the wrap. It is anticipated that adjusting the pH of the antimicrobial wrap to pH 6-8 may also produce a beneficial decrease in inflammation at the site, since for example acidic eluents may promote inflammation in surgical pockets such as, e.g., breast reconstruction pockets. pH may be adjusted in an antimicrobial wrap comprising minocycline by a variety of methods; for example, the pH may be adjusted in a bioactive spray solution applied to the wrap, or the pH may be adjusted by separately spraying and impregnating an alkaline solution to neutralize the acidic Minocycline in situ. In some embodiments, the wrap or biodegradable film has a pH of about 4-6 or 4-7 (e.g., 4, 4.5, 5, 5.5, 6, 6.5, 7, or any range derivable therein). For example, wraps that contain minocycline and are pH of 4-6 or 4-7 may exhibit improved stability, such as reduced degradation of the minocycline over time. The wrap or biodegradable film may be dehydrated or freeze dried before storage and prior to use.

One or more additional bioactive or therapeutic compound may be comprised in the antimicrobial wrap or bioactive film. The bioactive or therapeutic compound may be an antioxidant, hygroscopic agent, epimer-stabilizer, or a buffering or elution modifying agent. For example, the bioactive or therapeutic compound may be ascorbic acid, gentisic acid, a vitamin, a sugar (e.g., lactose or mannose), a moisturizer, a buffer, a chelator, salts such as magnesium sulfate, a hydrate, a protein, a peptide, a carbohydrate, a cytokine, a pain modulating agents (e.g., a local anesthetic), an anti-inflammatory agent (e.g., a NSAID), an antifibrotic, MeSNA, an enzyme, or a protease inhibitor.

In some embodiments, the film is wrapped around a medical implant or prosthesis, such as a breast implant, prior to insertion into a subject such as a human patient. The bioactive or antimicrobial films may display a melting temperature of less than 38° C.; thus, after insertion into the subject, the film may melt and release antimicrobial agents into the immediate vicinity of the implant. In this way, increased amounts of antimicrobial agents and/or additional therapeutics may be delivered around surfaces of an implant. Further, since all or part of the antimicrobial film may melt in situ within several minutes, e.g., from about 1 to less than 15 minutes, which may allow for a more thorough delivery of the antimicrobial agents to the surfaces of the implant as well as improved pharmacokinetics for release of the antimicrobial agents around the medical implant. The antimicrobial agents may reduce or substantially prevent infection resulting from a bacteria or fungi. In some embodiments the biodegradable antimicrobial film comprises a highly plasticized gelatin. In various embodiments, the antimicrobial film may be subjected to dehydrothermal treatment to increase the working time and/or toughness. The plasticizer content of a highly plasticized gelatin may be adjusted to increase ductility; as shown in the below examples, increased amounts of plasticizer (e.g., 31-60% glycerol) may be included in the highly plasticized gelatin to increase the ductility. In some embodiments, the films may contain multiple layers and/or regions comprising antimicrobial compounds and regions that do not contain antimicrobial compounds.

An aspect of the present invention relates to a biodegradable covering for a medical implant, the covering comprising a highly plasticized gelatin and at least one drug to reduce infection or capsular contraction, wherein the highly plasticized gelatin consists essentially of gelatin and from about 35% to about 60% plasticizer, wherein the plasticized gelatin has a melting temperature of less than 38° C., and wherein the biodegradable covering has been subjected to cryoprocessing or freeze-drying. The cryoprocessing or freeze-drying may comprise cooling the temperature to from about 0° C. to about −40° C., to from about −10° C. to about −25° C., or to from about −10° C. to about −20° C. The cryoprocessing may comprises dry air convection or applying dry air to the covering. The cryoprocessing or freeze-drying may occur for from 1 minute to 2 weeks, more preferably from about 1 hour to about 2 weeks. In some embodiments, the cryoprocessing occurs for about 1-24 hours. The freeze drying may comprise applying reduced atmospheric pressure to the covering. The reduced atmospheric pressure may result from a vacuum pump. The freeze-drying may occur for about 1-24 hours, or for about 1-8 hours. In some embodiments, the plasticized gelatin has a melting temperature of 27-37° C., 30-37° C., or any range derivable therein. In some embodiments, the plasticized gelatin comprises about 40-60% plasticizer. In some embodiments, the plasticizer is glycerol, a propylene glycol, a sugar, a carbohydrate, an amino acid, a salt, an acid, or a polyol. In some embodiments, the plasticizer is glycerol. In some embodiments, at least a portion of an inner surface of the covering is substantially sticky or adhesive, and a portion of or substantially all of an outer surface of the covering is substantially lubricious. In some embodiments, at least a portion of a surface of the covering has been treated with a gluconic acid solution. In some embodiments, at least a portion of a surface of the covering has been treated with a glycerol-gelatin liquid comprising about 60-90% glycerol or a solution comprising a carbohydrate, a starch, or a sugar. The covering may be sufficient in size or shaped to cover a breast implant. The covering may be shaped as a film, a wrap, a pouch or a bag. In some embodiments, the covering is a pouch or a bag; wherein the covering has a central region and a plurality of lateral appendages, or the covering is substantially star-shaped. The covering may comprise a plurality of biodegradable layers. In some embodiments, the at least one drug is selected from the group consisting of an antimicrobial agent, an anti-inflammatory agent, an anti-scarring agent, a hemostatic agent, an anti-neoplastic agent, a calcium channel blocker, a leukotriene inhibitor, an antifibrotic agent, a fibrotic agent, an anesthetic, an analgesic, and thrombolytic agent. The at least one drug may be comprised in a fiber, a bead, a particle, a liposome, a microsphere, or a nanosphere. In some embodiments, the at least one drug is an antimicrobial agent (e.g., bacitracin, cephalexin, gentamicin, an antiseptic, a chelator, chlorhexidine, gendine, gardine or mixtures thereof). In some embodiments, the antiseptic is hydrogen peroxide, chlorhexidine, gendine or gardine. The covering may further comprises mercaptoethane sulfonate (MeSNA), minocycline, rifampin, and/or glyceryl trinitrate (GTN). The covering may further comprise nitroglycerin or a nitric oxide donor. In some embodiments, the at least one drug is a leukotriene inhibitor (e.g., a leukotriene receptor antagonist selected from the group consisting of acitazanolast, iralukast, montelukast, pranlukast, verlukast, zafirlukast, and zileuton). In some embodiments, the covering comprises one, two, three, or all of mercaptoethane sulfonate (MeSNA), minocycline, rifampin, or glyceryl trinitrate (GTN). In some embodiments, the antimicrobial agent is minocycline. In some embodiments, the covering comprises minocycline and rifampin. The covering may have a pH of about 3-9, about 6-8, or about 7-7.4. In some embodiments, the covering comprises minocycline, rifampin, and mercaptoethane sulfonate. The covering may further comprise glyceryl trinitrate (GTN). The covering may further comprise a fatty acid or monoglyceride. The fatty acid may be a C₆₋₁₂ alkanoic acid or a C₆₋₁₀ alkanoic acid. In some embodiments, the fatty acid is hexanoic acid, octanoic acid, decanoic acid, dodecanoic acid, caprylic acid (octanoic acid), caproic acid, or lauric acid. In some embodiments, is caprylic acid (octanoic acid). In some embodiments, the covering comprises glyceryl trinitrate (GTN) and capyrilic acid. In some embodiments, at least a portion of the covering has been exposed to crosslinking. In some embodiments, at least half of the covering has been exposed to crosslinking. The crosslinking may comprise exposing at least a portion of the covering to radiation or to a dehydrothermal heat treatment. The crosslinking may be a mild or partial crosslinking. The crosslinking may be sufficient to increase the working time, toughness, or stiffness of the covering. The portion may comprise an antimicrobial agent (e.g., minocycline, rifampin, chlorhexidine, gendine, or gardine). In some embodiments, the portion comprises minocycline and rifampin. The portion may further comprise mercaptoethane sulfonate (MeSNA), glyceryl trinitrate (GTN), or a C₆₋₁₀ alkanoic acid (e.g., caprylic acid). The covering may comprise regions that have been exposed to crosslinking and regions that have not been exposed to crosslinking. In some embodiments, the regions that have not been exposed to crosslinking comprise the drug, and wherein the regions that have been exposed to crosslinking do not comprise the drug. In some embodiments, both the regions that have not been exposed to crosslinking and the regions that have been exposed to crosslinking both comprise the drug. In some embodiments, the regions that have not been exposed to crosslinking comprise the drug, and wherein the regions that have been exposed to crosslinking do not comprise the drug. The regions that have not been exposed to crosslinking may comprise minocycline and rifampin. The regions that have not been exposed to crosslinking may further comprise glyceryl trinitrate (GTN), mercaptoethane sulfonate (MeSNA), or caprylic acid. In some embodiments, at least a portion of the covering has not been exposed to crosslinking. The covering may comprise or consist of a single layer. The covering may comprise regions that have been exposed to crosslinking and regions that have not been exposed to crosslinking. The drug may be comprised in the regions that have not been exposed to crosslinking. The drug may be comprised in the regions that have been exposed to crosslinking. In some embodiments, the regions that have not been exposed to crosslinking are present in the covering in a pattern of shapes or in a sponge-like pattern. The shapes may comprise a plurality of substantially circular or oval shapes. In some embodiments, the covering has multiple layers. The covering may have 2 layers or at least 2 layers. In some embodiments, a layer has been exposed to crosslinking. The layer may comprise an antimicrobial agent. In some embodiments, the layer has been exposed to a dehydrothermal heat treatment and subsequently contacted with a solution containing the antimicrobial agent. In some embodiments, the layer is dried or exposed to a dehydrothermal heat treatment after being contacted with the solution. The solution may comprise an alcohol (e.g., ethanol or methanol) and water. The alcohol may comprise about 1-50% (v/v) of the solution. The solution may comprise gelatin and glycerol. In some embodiments, the covering comprises a first layer comprising a partially crosslinked plasticized gelatin and a second layer comprising a plasticized gelatin that has not been crosslinked, wherein the second layer comprises the drug. The second layer may comprise minocycline and rifampin. The highly plasticized gelatin may be comprised in an inner layer or a middle layer of the covering. In some embodiments, an outer layer of the covering has a melting temperature of greater than 38° C. In some embodiments, the covering has 3, 4, 5, or 6 layers. The covering may have 3 layers, wherein the 3 layers are an outer layer, a middle layer, and an inner layer. The outer layer, the inner layer, or the middle layer of the covering may comprise the drug. The middle layer may comprise the highly plasticized gelatin. The inner layer and/or the outer layer may have a melting temperature of greater than 38° C. In some embodiments, the outer layer and inner layer have been exposed to crosslinking. In some embodiments, regions of the middle layer have been exposed to crosslinking and regions of the middle layer have not been exposed to crosslinking, wherein said at least one drug is comprised in a least some of the regions that have not been exposed to crosslinking. One or all of the edges of the covering may be melted or welded together. In some embodiments, the covering comprises at least three layers, and wherein the edges of the outermost layers have been melted or welded together by the application of heat. In some embodiments, the outermost layers are partially crosslinked, and wherein an inner layer comprises the highly plasticized gelatin and the drug. The inner layer may comprise minocycline and rifampin. The application of heat may be via heat gun, food sealer, or laser. In some embodiments, the drug is an antimicrobial agent, and wherein the covering comprises a second drug. The second drug may be an anti-inflammatory agent, an anti-scarring agent, a hemostatic agent, an anti-neoplastic agent, a calcium channel blocker, a leukotriene inhibitor, a leukotriene inhibitor, an antifibrotic agent, a fibrotic agent, an anesthetic, an analgesic, or a thrombolytic agent. The covering may comprise minocycline and rifampin. The covering may further comprise glyceryl trinitrate (GTN), mercaptoethane sulfonate (MeSNA), caprylic acid or tranilast. The antimicrobial agent and the second drug may be comprised in overlapping regions of the covering. The antimicrobial agent and the second drug may be comprised on a surface of the covering. In some embodiments, the antimicrobial agent and the second drug are comprised or dispersed within the covering. In some embodiments, the antimicrobial agent and the second drug are comprised in non-overlapping regions of the covering. The antimicrobial agent and the second drug may be comprised on opposite sides of the covering. The highly plasticized gelatin may be comprised on an adhesive backing. The adhesive backing may be translucent. The adhesive backing may be part of a bandage or wound dressing. In some embodiments, the highly plasticized gelatin is translucent, and wherein bandage or wound dressing allows for viewing of skin or tissue under the bandage or wound dressing. The covering may be comprised on a backing. The backing may comprise silicone, a silicone coating, or PTFE. In some embodiments, the backing is further defined as a storage backing or a backing that can be removed prior use. In some embodiments, the pH of the covering is about 6-8 or about 7-7.4. In some embodiments, the pH of the covering is from about 1 to about 7, from about 4 to about 6, from about 4 to about 7, from about 1 to about 4, about 2-3, about 2.25-2.75, less than about 4, about 1-3, about 2-2.75, about 1-2.5, about 2.5 or less, less than about 2, or about 1, 1.5, 2, 2.5, 2.6, 2.7, 2.75, 2.8, 3, 3.5, 4, or any range derivable therein (e.g., pH 2.5-2.8). In some embodiments, the pH of the covering is from about 4 to about 7, from about 4 to about 6, or 4, 4.5, 5, 5.5, 6, 6.5, 7, or any range derivable therein. The covering may comprise rifampin and a tetracycline (e.g., minocycline). The pH of the covering may be from about 8 to about 12, or about 8, 9, 10, 11, 12, or any range derivable therein. In some embodiments, the covering has been substantially dehydrated or freeze-dried. It is anticipated that coverings that contain minocycline or have been impregnated with minocycline may display improved stability or shelf life (e.g., improved stability of the minocycline in the covering) over time when the pH is about 4-6 or about 4-7. The covering may be dehydrated or freeze dried prior to storage, and then rehydrated (e.g., using purified or deionized water) prior to use or insertion into a mammalian subject.

Another aspect of the present disclosure relates to a kit comprising a medical implant and the biodegradable covering as described herein or above. In some embodiments, the medical implant is a breast implant, a hernia mesh, a pacemaker stabilizing envelope, a gynecologic mesh, a neurologic or cranial overlay, a nerve guide (e.g., a spinal nerve guide), a tendon surgery implant, a periodontal implant, an oral-maxilofacial implant, nerve stimulator, implantable pump, ventricular assist device, anastomotic coupler, pin, rod, screw, soft tissue pledget or buttress, wire, or cable. The tendon surgery implant may be configured for use in a tendon surgery of the hand, foot, shoulder, or knee. The biodegradable covering may be freeze dried or dehydrated, and the biodegradable covering may be comprised in a container means comprising a moisture barrier material (e.g., aluminum foil, plastic, or glass). The container means may comprise a release lining film. The release lining film may be adjacent to or in physical contact with the biodegradable covering. In some embodiments, the release lining film is a paper liner, a silicone liner, or a polytetrafluoroethylene (PTFE) liner. The paper liner may comprise a silicone coating or a fluoropolymer coating. In some embodiments, the biodegradable covering has been sealed in the container means in (i) a substantially anhydrous environment and/or (ii) in a reduced oxygen or oxygen-free atmosphere. The kit may comprise an oxygen absorbing packet (e.g., an oxygen absorbing packet comprising iron powder) and/or a moisture absorbing packet (e.g., a moisture absorbing packet comprises a silica gel or an epoxy resin). In some embodiments, the biodegradable covering has been sterilized by exposure to electromagnetic radiation (e.g., comprising gamma radiation or E-beam radiation).

Yet another aspect of the present disclosure relates to a medical implant assembly comprising a biodegradable covering described herein or above containing the medical implant. The medical implant may be a breast implant, a hernia mesh, a pacemaker stabilizing envelope, a gynecologic mesh, a neurologic or cranial overlay, a nerve guide (e.g., a spinal nerve guide), a tendon surgery implant, a periodontal implant, an oral-maxilofacial implant, nerve stimulator, implantable pump, ventricular assist device, anastomotic coupler, pin, rod, screw, soft tissue pledget or buttress, wire, or cable. The tendon surgery implant may be configured for use in a tendon surgery of the hand, foot, shoulder, or knee. In some embodiments, the medical implant is a breast implant.

Another aspect of the present invention relates to a method for reducing at least one post-surgical indication from breast augmentation or breast reconstruction in a subject, the method comprising surgically implanting into the subject the breast implant assembly described above or herein. In some embodiments, the biodegradable covering is a film, and wherein the method comprising wrapping the breast implant with the biodegradable covering prior to insertion. The method further comprising trimming excess film prior to said implanting. The wrapping may occur prior to a surgery for the implantation. The wrapping may occur during a surgery that comprises the implantation. The indication may be selected from the group consisting of infection, inflammation, capsular contracture, adhesion, and scarring. In some embodiments, the biodegradable covering is used to line or cover part or all of a region in the subjects body, wherein the breast implant is subsequently placed on the biodegradable covering, and wherein the covering is subsequently used to cover the breast implant.

Yet another aspect of the present invention relates to a transcutaneous device assembly comprising a biodegradable covering described above or herein that is wrapped around at least a portion of the transcutaneous device. In some embodiments, the transcutaneous device is an electrical nerve stimulation device, a catheter, a screw, a rod, a pin, a wire, a collar, a tube, a surgical drain, a hernia mesh, a pacemaker stabilizing envelope, a gynecologic mesh, a neurologic or cranial overlay, a nerve guide, a tendon surgery implant, a periodontal implant, an oral-maxilofacial implant, nerve stimulator, implantable pump, ventricular assist device, anastomotic coupler, pin, rod, screw, soft tissue pledget or buttress, wire, or cable. In some embodiments, the transcutaneous device is a surgical drain.

Another aspect of the present invention relates to a method for reducing at least one post-surgical indication from implantation of a transcutaneous device in a subject, the method comprising surgically implanting into the subject the transcutaneous device assembly described above or herein. The subject may be a mammalian subject such as a human patient. In some embodiments, the portion of the transcutaneous device that is placed in the subject is covered by said covering. In some embodiments, the transcutaneous device is secured outside of the body of the subject with a wound dressing or bandage. The biodegradable covering may have a pH of less than about 4, less than about 3, about 2.5 or less, less than about 2, or about 1, 1.5, 2, 2.5, 2.6, 2.7, 2.75, 2.8, 3, 3.5, 4, or any range derivable therein (e.g., pH 2.5-2.8). The biodegradable covering may have a pH of less than about 4-7, about 4-6, or about 4, 4.5, 5, 5.5, 6, 6.5, 7, or any range derivable therein. The pH of the biodegradable covering may be raised to a pH of at least about 4 in situ by application of an alkaline solution to the biodegradable covering. In some embodiments, the pH of the covering is not adjusted or altered prior to the implanting. In some embodiments, the biodegradable covering has been dehydrated or freeze dried prior to application of the alkaline solution. The in-situ application may be performed prior to or just prior to the surgically implanting into the subject. The in-situ application may be performed prior to or just prior to the surgically implanting into the subject. The alkaline solution may have a pH of at least about 10, 11, 12, or any range derivable therein. The alkaline solution may comprise a second drug (e.g., an antimicrobial agent, an anti-inflammatory agent, an anti-scarring agent, a hemostatic agent, an anti-neoplastic agent, a calcium channel blocker, a leukotriene inhibitor, an antifibrotic agent, a fibrotic agent, an anesthetic, an analgesic, or a thrombolytic agent). In some embodiments, the biodegradable covering has a pH of from about 8 to about 12, at least 8, or about 8, 9, 10, 11, 12 or any range derivable therein. In some embodiments, the pH of the biodegradable covering is lowered to a pH of less than about 10 in situ by application of an acidic solution or a neutral solution to the biodegradable covering. In some embodiments, the biodegradable covering has been dehydrated or freeze dried prior to application of the acidic solution. The acidic solution may have a pH of about 1-4, about 4-7, or about 4-6. The in-situ application may be performed prior to the surgically implanting into the subject (e.g., during a surgical procedure on the subject). The in-situ application may be performed after the surgically implanting into the subject (e.g., the biodegradable covering may be placed in the subject and then pH of the biodegradable may be altered by application of a solution having a different pH than the biodegradable covering). The acidic solution may comprise a second drug (e.g., an antimicrobial agent, an anti-inflammatory agent, an anti-scarring agent, a hemostatic agent, an anti-neoplastic agent, a calcium channel blocker, a leukotriene inhibitor, an antifibrotic agent, a fibrotic agent, an anesthetic, an analgesic, or a thrombolytic agent).

Yet another aspect of the present invention relates to a method of producing the biodegradable covering described above or herein, comprising: (i) casting or melting the highly plasticized gelatin consisting essentially of gelatin and from about 35% to about 60% plasticizer to produce a film, (ii) subjecting the film to cryoprocessing or freeze-drying, and (iii) contacting the contacting with an aqueous solution comprising greater than 50% water and a drug, thereby coating or impregnating the film with the drug. The cryoprocessing or freeze-drying may comprise cooling the temperature to from about 0° C. to about −40° C., from about −10° C. to about −25° C., or from about −15° C. to about −20° C. The cryoprocessing may comprise dry air convection or applying dry air to the covering. The cryoprocessing or freeze-drying may occur for from about 1 hour to about 2 weeks (e.g., for about 1-24 hours). The freeze drying may comprise applying reduced atmospheric pressure to the covering. The reduced atmospheric pressure may be produced via a vacuum pump. In some embodiments, the method comprises using water droplet interfacial contact angles of 60-130 degrees. The method may comprise using water droplet interfacial contact angles of 75-90 degrees. In some embodiments, the biodegradable covering is applied to a removeable backing. The removeable backing may comprise a silicone coating, polytetrafluoroethylene (PTFE), a plastic, a coated plastic, parylene, or graphene. The covering may be sterilized with radiation (e.g., electron beam radiation, beta radiation, or gamma radiation). In some embodiments, the radiation is applied to the covering while the covering is maintained at a cryogenic temperature. The radiation may be applied to the covering while the covering is near or in contact with ice or dry ice. In some embodiments, the aqueous solution has a different pH than the film. In some embodiments, the aqueous solution alters the pH of the film resulting in a pH of about 6-8, or a pH of about 7-7.4, in the film. In some embodiments, the pH of the aqueous solution is about 2-3, about 1-4, less than about 4, less than about 3, less than about 2.5, or less than about 2, about 1-4, about 1-3, about 2-3, about 1, 2, 3, 4, or any range derivable therein, about 4-7, about 4-6, or about 4, 4.5, 5, 5.5, 6, 6.5, 7, or any range derivable therein. The aqueous solution may have a pH of about 1-3, about 2-3, about 1-2.5, or about 2.5-2.8 (e.g., 2.6, 2.7, 2.75, 2.8, or any range derivable therein). In some embodiments, the drug is rifampin or minocycline. The covering may comprise rifampin and a tetracycline (e.g., minocycline). In some embodiments, the aqueous solution has a pH of about 8-12. The aqueous solution may comprise an antimicrobial agent, an anti-inflammatory agent, an anti-scarring agent, a hemostatic agent, an anti-neoplastic agent, a calcium channel blocker, a leukotriene inhibitor, an antifibrotic agent, a fibrotic agent, an anesthetic, an analgesic, or a thrombolytic agent. In some embodiments, the film comprises minocycline and the pH of the film is about 4-6 or 4-7, and it is anticipated that this pH range of the film (about 4-6 or 4-7) may improve the shelf-life of the film (e.g., improve stability of the film in storage and/or improve the stability of the minocycline in the film in storage). The pH of the film may be adjusted to about 4-6 or about 4-7 prior to the cryoprocessing or freeze drying. For example, the film may be contacted with a second aqueous solution to result in a pH in the film of about 4-6 or about 4-7. The method may further comprise: (iv) subjecting the film to cryoprocessing or freeze-drying after step (iii); and (v) contacting the film with a second aqueous solution. The second aqueous solution may result in a pH of about 4-6 or 4-7 in the film. In some embodiments, the second aqueous solution is deionized water.

A variety of medical implants may be covered with or laminated with a biodegradable film of the present disclosure. For example, the medical device may be a breast implant, a penile implant, a cosmetic restorative or enhancement implant, an implantable prosthesis, or an orthopedic implant, a dental implant, an ophthalmic implant, a cranial implant, a cardiac implant, a pump, a regulator or a stimulator. In some embodiments, the implant is a hernia mesh, pacemaker stabilizing envelope, gynecologic mesh, neurologic or cranial overlay, nerve guide (e.g., a spinal nerve guide), implant for a tendon surgery (e.g., for use in a tendon surgery of the hand, foot, shoulder, or knee), periodontal implant, oral-maxilofacial implant, nerve stimulator, implantable pump, ventricular assist device, anastomotic coupler, pin, rod, screw, (such as a surgical pin, rod, or screw for an orthopedic or dental surgery), soft tissue pledget or buttress, wire, or cable. The film may be laminated onto or used to cover a portion of or all of the implant. In some procedures the biodegradable film containing an antimicrobial or bioactive agent is overlayed or place onto in to a surgical site following a cartilage or orthopedic surgeries/implants to prevent infectious or other complications following closure. The film may reduce or prevent adhesions, infections, fibrosis, inflammation or other procedural complications, and one or more bioactive agents to promote these effect(s) can be included in the film.

Antimicrobial agents included in the films and wraps as described herein may inhibit the growth of or kill a wide variety of genuses and species of bacteria and fungi including, e.g., spherical, rod-shaped, and spiral bacteria. Non-limiting examples of bacteria include staphylococci (e.g., Staphylococcus epidermidis, Staphylococcus aureus), Eterrococcus faecalis, Pseudomonas aeruginosa, Escherichia coli, among other gram-positive bacteria and gram-negative bacilli. Non-limiting examples of fungal organisms include Candida albicans and Candida krusei.

A variety of therapeutic compounds may be included in the biodegradable films as disclosed herein. These compounds include antibiotics; leukotriene antagonists, such as zafirlukast, montelukast, pranlukast and zileuton; antineoplastic agents, such as 5-fluoruricil; nitric oxide producing agents, such as L-arginine; calcium-channel blockers, such as verapamil; TNF; interleukins; interferons; paclitaxel or other chemotherapy agents; 2-mercaptoethanesulfonate; antifungal agents; as well as any other agent, especially those that are known to for their ability to reduce capsular contracture. Examples of non-steroidal anti-inflammatory agents include, but are not limited to, acetaminophen, aspirin, celecoxib, diclofenac, diflunisal, flurbiprofen, ibuprofen, indomethacin, ketoprofen, ketorolac, meclofenamate, meloxicam, methyl salicylate, nabumetone, naproxen, oxaprozin, piroxicam, sulindac, tolmetin and trolamine. Examples of antimicrobial drugs include, but are not limited to: aminoglycosides, such as amikacin, gentamicin, kanamycin, neomycin, streptomycin, and tobramycin; antibiotics, such as bacitracin, clindamycin, daptomycin, lincomycin, linezolid, metronidazole, polymyxin, rifaximin, vancomycin; cephalosporins, such as cephazolin or cephalexin; macrolide antibiotics, such as erythromycin, azithromycin and the like; α-lactam antibiotics, such as penicillins; quinolones, such as ciprofloxacin; sulfonamides, such as sulfadiazine; tetracyclines, such as minocycline and tetracycline; and other antibiotics, such as rifampin, triclosan, chlorhexidine, gendine, and gardine.

The phrase “a chelator” denotes one or more chelators. As used herein, the term “chelator” is defined as a molecule comprising nonmetal atoms, two or more of which atoms are capable of linking or binding with a metal ion to form a heterocyclic ring including the metal ion.

Unless noted otherwise, all percentages used herein refer to percent weight per weight (% w/w).

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Laminate wrap containing minocycline and rifampin.

FIG. 2: Cryoprocessed wrap.

FIG. 3: Wrap adhered to a glass surface.

FIG. 4: Wrap not able to maintain dimensional integrity.

FIG. 5: Cryoprocessed wrap containing minocycline and rifampin in discrete impregnated regions of the wrap.

### DESCRIPTION OF ILLUSTRATIVE EMBODIMENTS

In some aspects, flexible solid films with improved surface properties and/or improved handling properties are provided. The present disclosure is based, in part, on the observation that cryoprocessing or freeze-drying of antimicrobial wraps or biodegradable films comprising a highly plasticized gelatin (containing gelatin and about 30-60% plasticizer, more preferably about 40-60% plasticizer) resulted in wraps with superior surface and handling properties that may facilitate clinical use, e.g., in surgical procedures. The films may contain one or more antimicrobial or therapeutic agents and can be wrapped around a medical implant or device prior to implantation in a mammalian subject. After implantation, the film can rapidly melt due to the temperature of the subject, e.g., to form a conformal liquid coating around the implant or device. The film may be shaped into a bag, a pouch, or a covering into which the device is inserted prior to implantation. The film may substantially melt or liquefy within minutes after implantation, e.g., about 5-20 minutes, due to the melting temperature of the film. The film generally requires sufficient mechanical strength to be able to withstand the wrapping and implantation steps without fracturing. The film can contain antimicrobial agents, analgesic agents, anti-scarring agents, anti-inflammatory agents and/or anti-fibrotic agents. The antimicrobial agents may be encapsulated in fibers or microspheres in order to extend their longevities around the implant. The film and encapsulating agents are preferably bioabsorbable. The film may be coated with an adhesive layer on one or both sides of the film. In some embodiments, the film is layered such that one side of the film is sticky or adhesive, and may facilitate adherence to the medical device, and the other side is lubricious to facilitate implantation into a tissue pocket.

Using a biodegradable antimicrobial covering or film that liquefies in situ after insertion into a mammalian subject may provide several advantages. For example, in some embodiments, such a covering may provide improved comfort immediately following implantation. A liquid coating would generally not present edges that could be irritating to soft tissues. In contrast to a solid cover which could tear or create friction from physically shifting positions within around the implant during healing, an implant that liquefies in situ after insertion may be able to substantially move within is local environment cover or alternatively the implant would not be impeded. This may be particularly important for tissue expander implants such as breast implants where the shape of the implant is changed in situ over time. Unlike previous solid shaped conformal pouches, such as those described in US20080241212, that require the manufacture of different sized solid pouches to accommodate different size devices, the liquefying films as provided herein may be produced in a single size to accommodate a wide variety of devices, e.g., by either trimming the film at the point of use or by overwrapping to form a thicker liquid coating. Applying the liquid coating as a solid for purposes of implantation can provide significant advantages, e.g., if a coating was applied as a liquid there would be a risk that it could spill off the side of the device or be scraped off or depressed into thin regions during manipulation in preparation for insertion or during the insertion process.

In some embodiments, the film or covering comprises a highly plasticized gelatin. The highly plasticized gelatin may be substantially or essentially nontoxic. The plasticized gelatin may provide advantages including, e.g., a relatively low cost, improved safety, and a predictable bioabsorption profile. In certain embodiments, the highly plasticized gelatin can be easily wrapped around an implant or tissue expanders and molded to their shape such that the device can be inserted with a conformal wrap. The wrap may melt in-situ within minutes providing a conformal liquid coating that can deliver antimicrobial (as well as other) medications to substantially all surfaces of the implant.

### I. BIOABSORBABLE PLASTICIZED POLYMERS

In some embodiments, a biodegradable film or covering of the present invention comprises a bioabsorbable plasticized polymer such as, e.g., a highly plasticized gelatin. Generally, the films have a melting temperature such that they are substantially solid at room temperature, but will melt or liquefy after insertion into a mammalian subject, such as a human patient.

In some embodiments, the bioabsorbable plasticized polymer is a highly plasticized gelatin. Gelatins are protein based colloid solutions that tend to have a defined shape and allow for some movement, but typically they may be easily broken with mechanical force. In some embodiments, the strength of a gelatin is increased by introduction of a plasticizer, such as glycerol. In some embodiments, a highly plasticized gelatin may be produced as described in U.S. Pat. No. 3,042,524 or U.S. Pat. No. 5,622,740, which are incorporated by reference herein in their entirety. The plasticizing agent can increase the strength of the film and allow for the modulation of the melting temperature. In some embodiments, the addition of plasticizing agents can be used to reduce the melting temperature (Tₘ) of a plasticized gelatin to less than 38° C. (e.g., 21-38° C., 25-37.05° C., 29-37° C., etc.).

Plasticized gelatins are distinct and different from gelatin. Plasticized gelatin is displays different physical properties as compared to gelatin, including increased mechanical strength. The form of plasticized-gelatin taught in U.S. Pat. No. 5,622,740 (containing 5-30% plasticizer) is suitable for use as food casings while ordinary, non-plasticized gelatin would have been too weak and susceptible to cracking. In contrast to gelatin, plasticized-gelatin can be processed with conventional extrusion equipment. The use of conventional extrusion equipment may also provide economic advantages, as compared to gelatin, since this equipment can be used to manufacture large coverings or films.

In some embodiments, the plasticized gelatin is a highly plasticized gelatin containing a plasticizer concentration range of from greater than about 30% to about 60%. Highly plasticized-gelatin can display sufficient strength while in solid form to wrap a medical implant such as a breast implant, an ability to rapidly melt once implanted, and/or an ability to wrap and conformally adhere to a medical device. In some embodiments, the plasticized-gelatin taught in U.S. Pat. No. 5,622,740, which contain 5-30% plasticizer, are not used since these plasticized gelatins would be too stiff to wrap and conformally adhere to a medical device without some additional device such as a clip, suture or staple to secure it and prevent unwrapping. The plasticizer included in the highly plasticized gelatin may be, e.g., glycerol, a propylene glycol, a sugar, or a polyol.

Other bioabsorbable polymers with an appropriate melting temperature range may be used in various embodiments. For examples, the bioabsorbable polymer may be a caprolactone based polymer or copolymer, or a trimethylene carbonate polymer or copolymers. In other embodiments where irritation to a subject is a concern, caprolactone polymers and trimethylene carbonate polymers may be avoided, as they can degrade in vivo into acidic moieties that may cause irritation. In some embodiments, the bioabsorbable polymer may be a polyphosphazine or amino-acid based polymers. Plasticizers for these polymers include DMSO, benzyl benzoate, glycol furol, and N-methyl pyrrolidone. Certain starch and cellulose-based polymers may also be used in various embodiments. In some embodiments, the bioabsorbable polymer is a plasticized protein or polypeptide. The plasticized proteins or polypeptides may be used for forming a convertible solid film. In some embodiments the film can comprise a solid wax. In some embodiments, meltable wax compositions do not include substantial quantities of lipid-based polyols that can be metabolized to acidic moieties that become irritating inside the body; for example, Trilucent™ oil filled breast implants caused complications resulting by lipid metabolism, and were removed from the market as a result of inflammatory complications associated with metabolic conversion of lipids that leaked outside of the silicone rubber envelope of the implants. In some embodiments, the film may comprise a fatty acid such as caprylic acid. As shown herein, fatty acids such as caprylic acid may be included in a film, e.g., at a concentration of less than about 10%, to improve the antimicrobial properties of the film. fatty acids such as caprylic acid may be included in a film or antimicrobial wrap of the present invention in an amount of, e.g., less than about 10%, less than about 5%, 0.01-10%, 0.01-5%, 0.1-5%, 0.5-10%, 0.1-9%, 1-8%, 1-7%, 1-6%, or 1-5%.

Additional bioabsorbable plasticizer-polymer combinations that may be used in various embodiments are listed below.

Other Bioabsorbable Plasticizer-polymer combinations
that may have Tm < 38° C.:

| BioAbsorbable Polymer | Plasticizers |
|---|---|
| Poly lactide coglycolide | DMSO, n-METHYL 2-PYRROLIDONE, |
| | tetraglycol, glycol fural, propylene |
| | carbonate, triacetin, ethyl acetate, benzyl |
| | benzoate |
| Poly Caprolactone coglycolide | Same as above |
| Poly Caprolactone colactide | Same as above |
| Poly Dioxanone coglycolide | Same as above |
| Poly Caprolactone cotrimethylene | Same as above |
| carbonate | |

### II. PLASTICIZERS

A variety of plasticizing agents may be used in various embodiments of the present disclosure, e.g., to alter the physical properties of and/or reduce the melting temperature of a bioabsorbable plasticized polymer. For example, plasticizing agents such as aliphatic polyols, poloxamers, sugars, and polyethylene glycols are contemplated for use in the bioabsorbable highly plasticized polymers. The plasticizer may be an amino acid or a carbohydrate. In some embodiments, the plasticizing agent is glycerol. In some embodiments, the polyols of the formula:

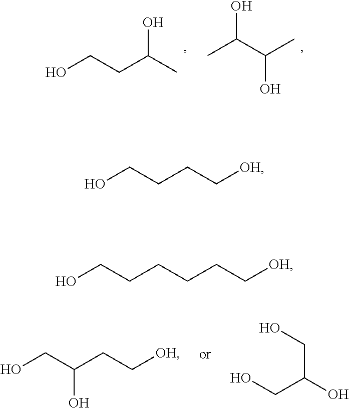

may be used. In some embodiments, the highly plasticized gelatin may arise from the combination of porcine gelatin and glycerol together. In some embodiment, the plasticizing agent can be used in percentages of approximately 30-60% of the bulk material.

As used herein, the term “highly plasticized” refers to the inclusion of from greater than about 30 to about 60% of a plasticizer in a bioabsorbable polymer. Various ranges of plasticizer may be included in a bioabsorbable polymer such as, e.g., 31-60%, 35-60%, 40-60%, or 35%, 40%, 45%, 50%, 55%, 60%, or any range derivable therein.

### III. SURFACE PROPERTIES

In some aspects, it has been observed that freezing or freeze-drying the antimicrobial film or biodegradable wrap containing a highly plasticized gelatin (comprising gelatin and about 30-60% plasticizer) can result in wraps with superior handling properties, such as decreased tackiness without the creation of voids in the wrap or film. As described herein, the decreased tackiness and improved surface properties can significantly aid in the clinical use or application of the wrap or film during a surgical procedure. For example, the decreased tackiness may reduce undesirable sticking of the wrap or film to either the surface of a liner or package where the wrap is contained and/or to gloves or any surgical instruments that are used to handle the wrap, while still allowing for a wrap or film that has sufficient flexibility to cover or wrap around a portion or all of an implant that is inserted into a mammalian subject during a surgery.

Various methods of freezing or freeze-drying can be applied to the antimicrobial film or biodegradable wrap. For example, the wrap may be stored in a freezer or exposed to low temperature (chilled) surfaces, radiantly cooled, exposed to low temperature convected fluids, or any combination thereof to decrease the temperature of the wrap. In some embodiments, the wrap or film containing the highly plasticized gelatin may be cooled to from about 0° C. to about −40° C., from about 0° C. to about −30° C., from about −5° C. to about −30° C., from about −10° C. to about −30° C., from about −15° C. to about −25° C., from about −10° C. to about −20° C., or about 0,−5,−10,−15,−20,−25,−30,−35,−40,−45,−49, or any range derivable therein. The wrap or film may be maintained at this temperature for at least about 1-3 hours, 1-6 hours, 1-12 hours, 1-24 hours, 1 day, 1-3 days, 1-6 days, 1 week, 2 weeks, or 1, 2, 3, 4, 5, 6, 7, 8, 9, 10, 11, 12, 13, 14 days, or more, or any range derivable therein. If desired, the wrap or film can be subjected to a continuous or intermittent airflow (e.g., using dry air convection) during cooling and/or when the wrap of film has been cooled. Optionally, the wrap or film may be subjected to reduced environmental pressure (e.g., using a vacuum pump) when the wrap or film is being cooled or while the wrap or film is at the reduced pressure.

After the film or wrap is subjected to the reduced temperature under conditions that allow for sublimation of water from the film or wrap, the film or wrap can then be hermetically sealed to reduce or prevent additional sublimation, and in this way it is anticipated that the film or wrap can be maintained in a frozen state for as long as desired (e.g., until just prior to surgical use). If the wrap or film is hermetically sealed, it is anticipated that there is no limit on the time the wrap might be maintained in the frozen state if the water is prevented from sublimating.

In some embodiments, the wrap of film is subjected to freeze drying. For example, the wrap of film can be placed in a freeze dryer at the desired temperature (e.g., from −10° C. to about −20° C.), and sublimation can be facilitated by exposure to very low environmental pressure produced by a vacuum pump. The cryoprocessed sublimated film or wrap can be removed after the desired period of time (e.g., about 12-24 hrs, 1-2 days, etc.). As shown in the below examples, similar benefits to the surface properties of the film or wrap can be achieved using either freeze drying or cooling with dry air convection to promote sublimation.

It is anticipated that other methods for cooling, such as cooling in dry ice or other very cold solutions, can be employed; when dry ice is used it is anticipated that insulation may be included around to wrap to prevent the wrap from getting colder than desired, or the wrap could be exposed to the dry ice for a relatively brief time prior to allowing the wrap allowed to warn to the desired temperature range. Generally, reducing the temperature below the glass transition temperature risks pores forming or the wrap cracking or breaking. It is anticipated that if the temperature is dropped briefly below the glass transition temperature of the wrap, then the wrap should be allowed to thaw to above the glass transition temperature in order to minimize the wrap cracking or breaking. In some embodiments, it is anticipated that a heated gel-dryer system can be used to cool the wrap or film. In some preferred embodiments, a large-scale freeze drier is used to cool the wrap or film to the desired range, since such commercially available freeze driers can allow for less complicated and more practical methodologies.

Although a variety of temperatures may be used when freezing or freeze-drying the antimicrobial wrap or biodegradable film, in some preferred embodiments a temperature is selected that is at or below the freezing temperature of water (0° C., or below) and is also above the glass transition temperature of the wrap or film (e.g., a freezing temperature that is above about −50° C. when using a wrap or film that contains about 65% by weight of a highly plasticized gelatin may be used in order to maintain the temperature above the glass transition temperature). Without wishing to be bound by any theory, it is anticipated that cooling the wrap or film to a temperature that is below the glass transition temperature may result in the undesirable creation of a porous structure in or voids in the film or wrap. It may also be desirable to cool the wrap or film to a temperature that is both at or below the freezing temperature of water and above the freezing temperature of the solvent solution (e.g., containing water and glycerol) contained within the wrap or film. As would be understood by one of skill, the freezing temperature of a water and glycerol mixture or solution is dependent on the concentration of the different components. The glass transition temperature of the wrap or film could possibly be different (e.g., slightly different) than the freezing temperature of the solvent mixture plasticizing the wrap. For example, at 30% by weight of glycerol, the freezing temperature of a water and glycerol mixture is approximately −10° C. In some embodiments, the wrap is cooled to a temperature that is both (i) below the freezing temperature of water, and is also (ii) above the glass transition temperature of the wrap and above the freezing temperature of the solvent mixture. In some embodiments, it is anticipated that colder temperatures within this range may slow the sublimination of solvents during the process, which may be undesirable or more inefficient when more rapid production methods are desired. Without wishing to be bound by any theory, it is anticipated that cooling or freezing the wrap to below its glass transition temperature could reduce or prevent sublimation, could reduce beneficial effects on the surface properties of the wrap or film (e.g., if, after the freezing and upon raising the temperature, the film returns to a state that is very similar of substantially the same as its state prior to the freezing). Without wishing to be bound by any theory, it is anticipated that sublimation that occurs during the cooling (e.g., within the ranges described above and herein) may contribute to the beneficial improvements in the surface and handling properties observed for the wraps and films. The freeze drier may, for example, cool the entire chamber by convection, similar to a conventional freezer. The freeze drier may run coolant through the shelves (not convection) and cool by conduction (contact). It is anticipated that either of these freeze drier methodologies can be used in aspects of the present disclosure to cool a film or wrap as described herein.

### IV. PH OF THE FILMS AND WRAPS

In some aspects, the antimicrobial wrap or biodegradable film containing the highly plasticized gelatin may be adjusted to a pH of about 6-8 or about 7.2-8, if desired. For example, adjusting the pH of the antimicrobial wrap may be particularly useful or advantageous when the antimicrobial wrap comprises minocycline. Although intravenous minocycline is normally administered at about pH 4 in order to stabilize the minocycline, and as shown in the below examples, raising the pH of antimicrobial wraps containing minocycline to a pH of about 6-8 was achieved without any observed decrease in the antimicrobial properties of the wrap. The antimicrobial wrap may include minocycline optionally in combination with another antimicrobial agent (e.g., rifampin), wherein the pH of the antimicrobial wrap has been adjusted to about pH 6-8. It is anticipated that antimicrobial wraps comprising minocycline that are adjusted to a neutral pH (or, e.g., pH 6-8, pH 7-7.4) may provide the advantage of reduced inflammation, as acidic eluents may potentially promote inflammation in surgical pockets (e.g., breast reconstruction pockets).

As shown in the below examples, impregnation of the antimicrobial wraps and biodegradable films provided herein was observed to be conducive to adjusting the pH of the wrap or film, which may be of particular importance for minocycline loaded wraps. In contrast, intravenous Minocycline is typically infused intravascularly at pH 4 and the hydrochloride salt is formulated to create a solution pH of about 4 when dissolved in aqueous infusates, in order to stabilize the minocycline.

Adjusting the pH of an antimicrobial wrap comprising minocycline may be achieved as follows. The pH of the wrap can be adjusted to about pH 6-8, minocycline can be impregnated into wraps, and then the antimicrobial wrap may be cooled or cryoprocessed as described herein in order to promote sublimation and improve the surface or handling properties as described herein. The minocycline impregnated wrap can be sublimated while at a neutral pH or a desired pH (e.g., pH of 6-8, pH 7.2-8, or pH 7-7.2) and then retain the neutral pH or the desired pH (e.g., pH of 6-8, pH 7.2-8, or pH 7-7.2) solution when reswollen in saline.

pH can be adjusted in an antimicrobial wrap, for example, either directly in the bioactive spray solution or by separately spraying and impregnating an alkaline solution to neutralize the acidic agent (e.g., minocycline) in situ. It is possible to incorporate other bioactive or protective molecules in using these approaches including, e.g., antioxidants, hygroscopic agents, epimer-stabilizers, and buffering or elution modifying agents. Examples include ascorbic acid, gentisic acid, vitamins, sugars (e.g., lactose, mannose, etc.), moisturizers, buffers, chelators, salts such as magnesium sulfate, hydrates, proteins, peptides, carbohydrates, cytokines, pain modulating agents, anti-inflammatory agents, antifibrotics, MeSNA, enzymes, and inhibitors of proteases (e.g., MeSNA) that may driving bioabsorption of the wrap (Rosenblatt et al., 2017).

In some embodiments, impregnation of different antimicrobial agents into an antimicrobial wrap or biodegradable film is achieved by the sequential application of solutions containing different antimicrobial agents and having different pH. For example, one or more antimicrobial agents (e.g., rifampin and/or minocycline) dissolved in a solution having an acidic pH of about 2.5-3 (pH of about 2.5, 2.6, 2.7, 2.75, 2.8, 3, or any range derivable therein) or less can be applied to a wrap or film as described herein to impregnate the one or more antimicrobial agents into the wrap or film, and then an alkaline solution is applied to the wrap or film to adjust the pH of the wrap of film to at least about 4. The wrap or film may then be dehydrated or freeze dried under vacuum prior to storage. Prior to use, the wrap or film may be rehydrated prior to use (e.g., rehydrated with deionized water), and this rehydration may alter the pH of the wrap or film, resulting in a pH of about 4-6 or 4-7 in the wrap or film.

In some embodiments, multiple therapeutic or antimicrobial agents (e.g., minocycline and rifampin) are comprised in an acidic solution (e.g., having a pH of about 2.5, 2.6, 2.7, 2.75, 2.8, 3, or any range derivable therein) that is applied to a wrap or biodegradable film described herein to impregnate the multiple therapeutic or antimicrobial agents (e.g., minocycline and rifampin) into the wrap or film. The pH of the wrap or film can then be adjusted to about 4 or greater by application of an alkaline solution (e.g., comprising NaOH), a neutral solution, or water (e.g., deionized water). The wrap or film may then be dehydrated or freeze dried under vacuum prior to storage. Prior to use, the wrap or film may be rehydrated prior to use (e.g., rehydrated with deionized water), and this rehydration may alter the pH of the wrap or film, resulting in a pH of about 4-6 or 4-7 in the wrap or film. The wrap or film may be rehydrated prior to of after insertion into a mammalian subject.

In some embodiments, a first therapeutic or antimicrobial agent (e.g., rifampin) is comprised in an acidic solution (e.g., having a pH of about 2.5, 2.6, 2.7, 2.75, 2.8, 3, or any range derivable therein) that is applied to a wrap or biodegradable film described herein to impregnate the first therapeutic or antimicrobial agents into the wrap or film. A second therapeutic or antimicrobial agent (e.g., minocycline) comprised in a second aqueous solution (e.g., having a pH of at least about 4 or higher, or a pH of 4, 5, 6, 7, 8, 9, 10, 11, 12, or any range derivable therein) is applied to a wrap or biodegradable film described herein to impregnate the second therapeutic or antimicrobial agent into the wrap or film. In some embodiments, the second aqueous solution has a pH of about 3.5-4.5 (e.g., 4) and comprises minocycline. The wrap or film may then be dehydrated or freeze dried under vacuum prior to storage. Prior to use or after insertion into a mammalian subject, the wrap or film may be rehydrated (e.g., rehydrated with deionized water), and this rehydration may alter the pH of the wrap or film, resulting in a pH of about 4-6 or 4-7 in the wrap or film.

In some embodiments, a wrap or biodegradable covering described herein may have a pH of from about 4 to about 7, from about 4 to about 6, or 4, 4.5, 5, 5.5, 6, 6.5, 7, or any range derivable therein. The wrap or biodegradable covering may be impregnated with antimicrobial agents (e.g., minocycline and/or rifampin) in an acidic solution (e.g., having a pH of about 2-4, or about 2-3) and then freeze dried prior to storage. The wrap or biodegradable covering may then be rehydrated prior to use of after insertion into a mammalian subject (e.g., using deionized or purified water). The pH of the freeze dried wrap may optionally be raised to a pH of about 4-6 or a pH of about 4-7 prior to use or after insertion into a mammalian subject. In some embodiments, the pH of the wrap or biodegradable covering is not adjusted in situ or just prior to use (e.g., prior to implantation into a mammalian subject or patient). In some embodiments the wrap or biodegradable covering is adjusted to a pH of of from about 4 to about 7, from about 4 to about 6, or 4, 4.5, 5, 5.5, 6, 6.5, 7, or any range derivable therein prior to freeze drying or storage. It is anticipated that wraps or biodegradable coverings that have been impregnated with minocycline and adjusted to a pH of about 4-6 or 4-7 may display superior storage properties, such as for example improved stability of minocycline in the wrap or biodegradable film over time.

In some embodiments, the pH of the antimicrobial wrap (e.g., comprising minocycline) or biodegradable wrap is adjusted to a pH of about 3-9, 4-8, 5-8, 6-8, 7-8, 4-7, 4-6, 5-7, 5-6, 6-8, 7-7.4, 7-7.2, 7-7.6, 7.2-7.6, 7.4-7.6, or about 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, or any range derivable therein.

### V. MELTING TEMPERATURES (T_{M}) OF ANTIMICROBIAL FILMS OR WRAP COMPOSITIONS

In contrast to solid biodegradable covers for medical implants that remain substantially solid or rubbery after insertion into a mammalian subject, biodegradable films or covers provided herein have, in various aspects, a melting temperature that allows for the biodegradable film or cover to remain substantially solid at room temperature (e.g., 15-25° C.), but liquefy after insertion into a mammalian subject.

This prior art does not anticipate a cover that is applied as a solid but rapidly converts into a liquid upon implantation. By rapidly we mean within a sufficient working time to implant a covered device once the implant site has been prepared. For a breast implant this typically requires several minutes.

In some aspects, the films or wrap compositions used herein may have a melting point of from about 23-36.5° C., about 24-37° C., about 25-37° C., about 30-37° C., or about 20, 21, 22, 23, 24, 25, 26, 27, 28, 29, 30, 31, 32, 33, 34, 35, 36, 37, or 38° C., or any range derivable therein. In addition to the melting temperature, the rate of liquefaction of the film or wrap (i.e., the rate at which the material liquefies) can also be affected by the degree of hydration of the material. For example, the wrap of film may require hydration for liquefaction; thus, if the wrap or film is more dehydrated (e.g., via a dehydrothermal heat treatment), then the film or wrap may hydrate more slowly and thus liquefy more slowly. The hydrophilicity of the plasticizer or the hydrophilicity of bioactive or antimicrobial agents present in the film or wrap may affect the degree of hydration and/or the rate of hydration of the material after inserted in a subject. Additionally, increased amounts of crosslinking, such as dehydrothermal heat treatment, can make the film tougher and slow the rate of liquefaction by removing more water from the film or wrap, thus slowing the liquefaction of the film or wrap after insertion into a subject, such as a human patient. In some embodiments, the melting or liquefaction of a film or wrap of the present invention may take at least 5, 10, 15, 20, 25, 30, 35, 40, 45, 50, 55, or at least 60 minutes after insertion onto or into a mammalian subject, such as a human patient.

The melting point (Tₘ) of a compound is distinct and different from the glass transition temperature (T_{g}) of a compound. There are several ways to describe the change in ordered structure of a compound and the temperature upon which that substance undergoes changes. The classic description of a melting temperature (Tₘ) relates specifically to the changing of a substance which goes from one phase to another, specifically from a solid phase to a liquid phase. On the other hand, the glass transition temperature (T_{g}) describes the conversion of an amorphous solid from a brittle solid into a more free flowing or rubbery glass. While a glass transition temperature can be near to the melting temperature, the glass transition temperature is almost always lower than the melting temperature. Finally, the glass transition temperature does not relate to a true phase transition like a melting temperature rather represents a series of different possible changes in properties such as viscosity of a polymer. Restated, although the glass transition temperature of the compound may be lower than 38° C., the melting temperature of the compound may not be below that threshold.

### VI. THERAPEUTIC AGENTS

One or more additional therapeutic or bioactive agent may be included in an antimicrobial film or biodegradable wrap provided herein, for example in addition to one or more antimicrobial agents (e.g., minocycline and rifampin). The therapeutic agent may be an antimicrobial agent, an anesthetic, an analgesic, an anti-inflammatory agent, an anti-scarring agents, an anti-fibrotic agent, a fibrotic agent (e.g., to promote anchoring), an anti-neoplastic agent, and/or a leukotriene inhibitor.

Therapeutic or bioactive agents may be incorporated into a film or cover of the present invention in a variety of ways. For example, one or more therapeutic agent may be dissolved or emulsified in the plasticizing liquids of the invention, e.g., to ensure a substantially even dispersal, and then the therapeutic agent(s) may be incorporated during the formation or synthesis of the film or cover. Alternatively, they could be suspended in a solid composition prior to forming and solidifying the films. In some embodiments, a therapeutic agent may be first encapsulated in fibers, beads, particles, liposomes, microspheres or nanospheres and then dispersed into a film or coating as described herein. In some embodiments, biodegradable microspheres, biodegradable nanospheres, or phospholipid liposomes may be utilized. The encapsulating polymers are preferably bioabsorbable. In some embodiments, the encapsulating polymers may degrade or absorb into the surrounding tissues at a different rates than the film, e.g., to prolong or reduce the rate of release of the therapeutic agent(s) into the surrounding tissues. The bioactive agent may also be applied as a thin mesh on top of or between film layers in a multilayer film by a variety of processes including nanospinning. Bioactive agents include antimicrobial agents, particularly combinations of minocycline and rifampin and other antimicrobials, gendine based combinations, and combinations of antimicrobials with nitroglycerin or nitric oxide donors. A chelator may be included in a bioabsorbable film of the present invention. Therapeutic agents that can be included in an antimicrobial wrap or biodegradable film provided herein include analgesic agents (e.g., lidocaine), an antiscarring agents (e.g., MeSNA), an anti-inflammatory agent (e.g., a steroid), an efflux pump inhibitor (e.g., Verapamil), or an antifibrotic agent (e.g., a TGF-beta inhibitor) antioxidants, hygroscopic agents, epimer-stabilizers, buffering or elution modifying agents, an analgesic, a local anesthetic, a volatile anesthetic, a pain medication or neuromodulator that is not an analgesic, Tranilast, an adhesion prevention agent (e.g., halofuginone), a callagenolytic agent, a bone forming (osteogenic) agent (e.g., a BMP, growth factor, or cytokine). and/or a moisturizer. In cases where anchoring is desired (e.g., a pacemaker envelope), a fibrotic agent can be included in the film or wrap. A clot inhibiting agent, a clot promoting agent, or a clot dissolving (thrombolytic) agent can be beneficially included in the film or wrap, depending on the type or surgery or condition being treated. In some embodiments, the bioactive agent is ascorbic acid, gentisic acid, a vitamin, one or more sugars (e.g., lactose, mannose, etc.), moisturizer, buffer, chelator, salt (e.g., magnesium sulfate), hydrate, protein, peptide, carbohydrate, cytokine, pain modulating agent (e.g., a local anesthetic, a volatile anesthetic, and analgesic agent), an anti-inflammatory agent (e.g., a NSAID), an antifibrotic agent, MeSNA, an enzyme, or a protease inhibitor (e.g., MeSNA). In some embodiments the antimicrobial wrap may contain both an antimicrobial agent (e.g., minocycline and rifampin) in combination with another bioactive agent for example as described above. In some embodiments, the bioabsorbable film includes one or more therapeutic agents or bioactive agents (e.g., anti-inflammatory agent, anti-scarring agent, anti-fibrotic agent, etc.), wherein the bioresorbable film does not contain an antimicrobial agent.

In some embodiments, a therapeutic agent as described in US20080241212, US20080128315, US20120052292, US20110082545, US20110082546, or US20120123535, which are incorporated herein in their entirety, may be included in a biodegradable film, pouch, sleeve, or covering, e.g., to for covering a breast implant, of the present invention.

The therapeutic agent may be an antimicrobial agent such as an ansamycin (e.g., rifamycin) and/or a tetracycline antibiotic (e.g., minocycline). In some embodiments, the bioabsorbable film comprises rifampin and minocycline. Inclusion of nitroglycerin or a nitric oxide donor, such as glyceryl trinitrate (GTN), may result in a synergistic enchantment of the antimicrobial or bactericidal effects of antibiotics (e.g., minocycline and rifampin). Inclusion of MeSNA of capyrilic acid may also result in a significant or synergistic improvement in the antimicrobial effects of minocycline and rifampin. The bioabsorbable film or covering may further comprise an antifungal agent or an antiviral agent.

In some embodiments, a nitroglycerin or nitric oxide donor is included in the bioabsorbable film. For example, the nitroglycerin or nitric oxide donor may be glyceryl tirnitrate (GTN), L-arginine, mono- or dinitrate (such as glycerol mono or dinitrate), nitrosocompound (such as nitrosoglutathione or nitrosocycteine), isosorbide nitrate (such as isosorbide di- or mono-nitrate), a nitroprusside, a diazenium diolate (such as NONOates), a nitric oxide complex (such as nitric oxide-spermine), or an exogenous nitric oxide generating catalyst (such as reduced silver, copper and other metal ions).

A variety of antibacterial agents may be included in the bioabsorbable film. The antimicrobial agent may be an antibacterial agent. Antibacterial agent that may be used include, e.g., aminoglycosides, beta lactams, quinolones or fluoroquinolones, macrolides, sulfonamides, sulfamethaxozoles, tetracyclines, streptogramins, oxazolidinones (such as linezolid), clindamycins, lincomycins, rifamycins, glycopeptides, polymxins, and lipo-peptide antibiotics. The antibacterial agent may be formulated, e.g., as a pharmacologically acceptable salt, in a lipid formulations, etc. Exemplary aminoglycosides that may be used in some specific aspects of the invention include amikacin, kanamycin, gentamicin, tobramycin, or netilmicin. Beta lactams are a class of antibacterials that inhibit bacterial cell wall synthesis. A majority of the clinically useful beta-lactams belong to either the penicillin group (penam) or cephalosporin (cephem) groups. The beta-lactams also include the carbapenems (e.g., imipenem), and monobactams (e.g., aztreonam). Inhibitors of beta-lactamase such as clavulanic acid and its derivatives are also included in this category. Non-limiting examples of the penicillin group of antibiotics that may be used in the solutions of the present invention include amoxicillin, ampicillin, benzathine penicillin G, carbenicillin, cloxacillin, dicloxacillin, piperacillin, or ticarcillin, etc. Examples of cephalosporins include ceftiofur, ceftiofur sodium, cefazolin, cefaclor, ceftibuten, ceftizoxime, cefoperazone, cefuroxime, cefprozil, ceftazidime, cefotaxime, cefadroxil, cephalexin, cefamandole, cefepime, cefdinir, cefriaxone, cefixime, cefpodoximeproxetil, cephapirin, cefoxitin, cefotetan etc. Other examples of beta lactams include mipenem or meropenem which are extremely active parenteral antibiotics with a spectrum against almost all gram-positive and gram-negative organisms, both aerobic and anaerobic and to which Enterococci, B. fragilis, and P. aeruginosa are particularly susceptible. Examples of beta lactamase inhibitors include clavulanate, sulbactam, or tazobactam. Exemplary macrolides include erythromycin, azithromycin, clarithromycin. Examples of quinolones and fluoroquinolones include nalidixic acid, cinoxacin, trovafloxacin, ofloxacin, levofloxacin, grepafloxacin, trovafloxacin, sparfloxacin, norfloxacin, ciprofloxacin, moxifloxacin and gatifloxacin. Exemplary sulphonamides include mafenide, sulfisoxazole, sulfamethoxazole, and sulfadiazine. The tetracycline group of antibiotics include tetracycline derivatives such as tigecycline, minocycline, doxycycline, demeclocycline, anhydrotetracycline, chlorotetracycline, and epioxytetracycline. The streptogramin antibacterial agents include quinupristin and dalfopristin. Other antibacterial drugs include glycopeptides such as vancomycin and teicoplanin. Other antibacterial drugs include polymyxins, such as colistin, prestinomycin, chloramphenicol, trimethoprim, fusidic acid, metronidazole, bacitracin, spectinomycin, nitrofurantion, daptomycin or other leptopeptides, oritavancin, dalbavancin, ramoplamin, and ketolide

A variety of chelators may be included in a bioabsorbable film as disclosed herein. Exemplary chelators include EDTA free acid, EDTA 2Na, calcium disodium EDTA, EDTA 3Na, EDTA 4Na, EDTA 2K, EDTA 2Li, EDTA 2NH₄, EDTA 3K, Ba(II)-EDTA, Ca(II)-EDTA, Co(II)-EDTACu(II)-EDTA, Dy(III)-EDTA, Eu(III)-EDTA, Fe(III)-EDTA, In(III-EDTA, La(III)-EDTA, CyDTA, DHEG, diethylenetriamine penta acetic acid (DTPA), DTPA-OH, EDDA, EDDP, EDDPO, EDTA-OH, EDTPO, EGTA, HBED, HDTA, HIDA, IDA, Methyl-EDTA, NTA, NTP, NTPO, O-Bistren, TTHA, EGTA, DMSA, deferoxamine, dimercaprol, zinc citrate, a combination of bismuth and citrate, penicillamine, succimer or Etidronate. The chelator may bind barium, calcium, cerium, cobalt, copper, iron, magnesium, manganese, nickel, strontium, or zinc.

### VII. IMPREGNATION OF THERAPEUTIC AND ANTIMICROBIAL AGENTS

A variety of methodologies for impregnating the therapeutic, bioactive, and/or antimicrobial agents into the biodegradable films and antimicrobial wraps are provided. In some aspects, the therapeutic or bioactive agent (e.g., an antimicrobial agent) can be impregnated into the biodegradable film by contacting the film (e.g., spraying, dipping, dripping, brushing, or spreading) with a aqueous solution comprising greater than about 50% water and the therapeutic or bioactive agent.

The bioactive wraps provided herein may be impregnated with one or more antimicrobial or bioactive agents dissolved in an aqueous solution containing greater than about 50% water. In some embodiments, reduced addition of water in the presence of the antimicrobial or bioactive agents is used to impregnate a wrap so less water would need to be removed post-impregnation or post-incorporation. For example, a highly plasticized blank gelatin wrap (not containing any bioactive or antimicrobial agents) can be produced via casting or laminating molten gelatin solutions, and subsequently the wrap is impregnated with the one or more bioactive agents (e.g., antimicrobial agents, anti-inflammatory agents, etc.) by spraying the wrap with an aqueous solution (containing greater than 50% water) the contains the one or more dissolved bioactive agents, thereby imbibing or impregnating by the wrap with the one or more bioactive agents and swelling the wrap.

In some embodiments, uniform impregnation can be attained by moving the spray nozzle uniformly across the surface of the wrap or by using an array of nozzles arranged to create a uniform spraying pattern. In some embodiments, the wrap is sprayed on both sides (e.g. simultaneously sprayed on both sides, sprayed on one side and subsequently sprayed on the other side) to obtain a substantially uniform impregnation of dissolved bioactive agents through the thickness of the wrap. Water miscible non-aqueous solvents can be included in the impregnating mixture to help solubilize bioactive agents. For example, an aqueous solution containing 5-20% (v/v) ethanol (e.g., 10% ethanol (v/v) and 90% water (v/v)) can be used when impregnating an antimicrobial agent (e.g., Rifampin) into a wrap. Other volatile water-miscible or emulsifiable organic and inorganic liquids can be included in the impregnation fluid to load bioactive agents of limited water solubility. Supercriticial fluids can also be used as a carrier for impregnating bioactive molecules, and the carrier fluid(s) or supercritical fluid can be removed post-impregnation, if desired. The impregnating fluid may contain one or more microemulsion, microsuspension, nanoemulsion, and/or nanosuspension, e.g., which may be convected with the impregnating fluid. Spraying can be performed using a pressurized gas (e.g., nitrogen, carbon dioxide, or other a volatile oxygen-free propellant) to reduce oxidative degradation of bioactive agent(s) and also performing the spray process in an oxygen-free (e.g., nitrogen gas) environment.

These methodologies can allow for incompatible bioactive agents (bioactive agents that cannot easily be impregnated using the same solution) to be separated spatially in a wrap or film. For example, one surface or (length×width) region of a wrap can be impregnated with a first bioactive agent, and subsequently a second bioactive agent (e.g., wherein the second bioactive agent is incompatible with the first bioactive agent or would not be applied using the same impregnation solution) is impregnated in a overlapping or non-overlapping surface or region of the wrap. Multiple agents, including both compatible bioactive agents and incompatible bioactive agents, can be included or impregnated in overlapping or non-overlapping regions of the film or wrap, as desired.

In some embodiments, impregnating the film or wrap with the bioactive or therapeutic agent in an aqueous solution containing greater than 50% water may allow for incorporating the bioactive agent while reducing (e.g., by greater than 50%) the amount of liquid that would later need to be removed from the film or wrap. Heat can be applied to remove excess fluid (water) from a wrap prior to impregnation. The subsequent incorporation by impregnation of the bioactive agents can thus require less water (aqueous carrier fluid) removal. Heat can be applied to the film or wrap prior to or after impregnation of a bioactive agent, e.g., to melt the film or wrap.

The aqueous solution containing the bioactive, antimicrobial, or therapeutic agent(s) preferably comprises greater than about 50% (v/v) water. In some embodiments, the aqueous solution comprises 55-99% (v/v) water, or at least 50, 55, 60, 65, 70, 75, 80, 85, 90, 95, 99, 99.5, 99.9, or 99.99% (v/v) water, or any range derivable therein.

A variety of temperatures of the aqueous solutions can be used. For example, these methods of impregnation of a wrap can allow for colder solutions (e.g., about 2-10° C.) to be used for impregnation. It is anticipated that use of colder aqueous solutions may slow or reduce oxidative and/or other degradation of bioactivity that may result from exposing delicate bioactive agents to higher temperature solutions (such as temperatures of a molten gelatin solution temperatures used in casting wrap layers). The inventors surprisingly found that chilled solutions when sprayed onto blank or unimpregnated wraps were rapidly and fully imbibed by the blank or unimpregnated wraps. In some embodiments, the temperature of the aqueous solution containing the bioactive or therapeutic agent is about 1-27, 2-26, 2-20, 2-15, 2-10° C., or 2, 3, 4, 5, 6, 7, 8, 9, 10, 11, 12, 13, 14, 15, 16, 17, 18, 19, 20, 21, 22, 23, 24, 25° C., or any range derivable therein.

In some embodiments, the therapeutic or bioactive (e.g., antimicrobial agent) can be impregnated in the wrap or film by mixing the therapeutic or bioactive agents (e.g., at high temperature) with the highly plasticized gelatin while casting the wrap, for example as described in U.S. Pat. No. 10,953,137.

### VIII. PROCESSING AND STORAGE

In some embodiments, the surface properties of equipment used to handle or produce the antimicrobial wraps and biodegradable films disclosed herein operate within certain processing contact angles in order to minimize tearing or the wraps or films.

Tearing due to tackiness of the wraps or films can occur during processing and forming of a film or wrap containing the highly plasticized gelatin (e.g., containing gelatin and 35-60% plasticizer) can be reduced or eliminated by utilizing improved surface properties of processing equipment. These surfaces can allow continuous or batch processing of highly plasticized gelatin wraps. As shown in the below examples, the affinity and adherence strength of highly plasticized gelatin wrap to surfaces with water droplet interfacial contact angles of 30 degrees or less were observed to be too great to be able to remove or peel away the highly plasticized wraps without tearing them. The high plasticization of the wraps can reduce the tensile strength such that they may tear when being held by a strongly adherent surface. The inventors have also found that the repulsion and lack of adhesion strength of highly plasticized gelatin wraps to surfaces with water contact angles of 160 degrees or more is too great to form consistent wraps with uniform thickness. The inventors observed that uniform wraps could be formed and removed from surfaces with contact angles ranging from 75 to 130 degrees; however, the 130 degree contact angle surface produced wraps with curling at the edges which was undesirable and requires trimming away the curled edges (insufficient adhesion strength to maintain wrap shape). As shown in the below examples, optimal surface contact angles for forming uniformly consistent highly plasticized gelatin wraps ranged from 75-90 degrees with a maximal possible range between 60 and 130 degrees.

In some embodiments, the antimicrobial wraps or biodegradable films containing a highly plasticized gelatin disclosed herein are produced or handled using equipment that operate at water droplet interfacial contact angles of from greater than 30 degrees to less than 160 degrees, more preferably from about 60-130 degrees, even more preferably about 75-130 degrees, about 75-90 degrees, or about 75, 80, 85, 90 degrees, or any range derivable therein.

In some embodiments a surface of the processing equipment that contacts the antimicrobial wrap or biodegradable film comprises or is coated with a non-stick material such as polytetrafluoroethylene (PTFE), a silicone, copolymers, blend, or derivative thereof.

In some embodiments, the antimicrobial wrap or biodegradable film is sterilized using irradiation. Impregnated wraps produced using the methods provided herein (e.g., including impregnation of bioactive or therapeutic agents in an aqueous solution and processing equipment operating within the contact angles described above) can be terminally sterilized by electron beam (beta) or gamma radiation, e.g., to provide extended shelf stability and to make the wrap amenable to surgical implantation. The radiation exposure can be performed while the wrap is maintained at cryogenic temperatures (for example on ice or dry ice) in order to reduce or prevent degradation of impregnated bioactive compounds. Prior to sterilization antimicrobial wraps formed via the method provided herein can be packaged, e.g., by placing the wrap or film on a liner and then vacuum sealing in a pouch or container (e.g., a foil pouch). Prior to vacuum sealing, a moisture absorbing packet (e.g., silica packets) can be added to the pouch or container, if desired, to scavenge residual moisture, and/or an oxygen scavenging packet (e.g., a packet comprising iron or activated carbon) can be included in the pouch or container.

The antimicrobial wrap or biodegradable film may be stored against a backing that has reduced adhesion properties. In some embodiments, the wrap or film is stored on a backing comprising or consisting of silicone (e.g., a silicone coating), PTFE, a plastic (e.g., an inert plastic, a coated plastic (e.g., coated with an inorganic compound such as a metal, mineral, or ceramic), parylene, or graphene. As shown in the below examples, silicone-coated or PTFE liners can provide easier release and less surface drug transfer than paper (parchment paper) liners.

### IX. PATTERNED AND/OR LAYERED FILMS AND COVERINGS

In some embodiments, an antimicrobial covering or film of the present invention comprises regions that contain antimicrobial compounds and regions that do not contain antimicrobial compounds. The antimicrobial covering or film may comprise 2, 3, 4, or more layers. In some embodiments, the antimicrobial covering or film may contain 2 or more layers, wherein some layers contain antimicrobial compounds and other layers do not contain antimicrobial compounds. For example, in some embodiments, the film may comprise three layers including two outer layers that do not contain antimicrobial compounds and a middle layer that contains one or more antimicrobial compound(s) (e.g., minocycline, rifampin, GTN, MeSNA, and/or caprylic acid; minocycline and rifampin; minocycline, rifampin, and GTN; minocycline, rifampin, and MeSNA; minocycline, rifampin, MeSNA, and caprylic acid) that are either continuously distributed throughout the middle layer or contained in regions of the middle layer. In some embodiments, the outer layers of a layered film may have either higher melting temperatures and/or improved handling properties. In some embodiments, it may be desirable to include the antimicrobial compound(s) the outer layers of a layered film or covering. As would be appreciated by one of skill in the art, the pattern of distribution of antimicrobial compounds in regions of a film or layer of film may be selected as desired; for example, the regions may be roughly circular or oval (e.g., in a “polka-dot” pattern), square, striped, etc., as desired. In some embodiments, an antimicrobial film or antimicrobial layer of film may contain the antimicrobial compounds distributed throughout the layer in a sponge-like pattern based on the creation of voids in the film that are subsequently filled with a filler (e.g., containing or consisting of a highly plasticized gelatin) comprising the antimicrobial compound(s).

In some embodiments, regions in a film that contain antimicrobial compound(s) may be introduced into the film, e.g., by removing portions of the film or creating voids in the film that are subsequently filled with a molten filler (e.g., a highly plasticized gelatin) that contains the antimicrobial compound(s). Different shaped and/or sized voids (e.g., windows, textures, sponge-like voids, etc.) may be created in or introduced into a film or layer of film (e.g., for films that include 2, 3, 4, or more layers) as desired. Several methods for creating voids in films that may be used to generate voids in a film that can subsequently be filled with molten bioactive fillers may be used with the present invention. For example, in some embodiments, fillers such as salts or sugars may be added to a film and subsequently dissolved away, leaving behind voids that may subsequently be filled with a composition (e.g., highly plasticized gelatin) containing the antimicrobial compound(s).

### ×. METHOD FOR INCREASING THE WORKING TIMES OF THE FILMS

In some cases it may be desirable to reposition implants comprising or covered in an antimicrobial film of the present invention following initial placement in the body of a subject or human patient. In some embodiments, it may be desirable for films to retain their solid properties for several minutes to as long as 1 hour to allow for implant repositioning prior to liquefying. As used herein, the “working time” of a film generally refers to the amount of time that the film may be handled for before it becomes substantially liquefied; thus, films may exhibit longer working times, e.g., by exhibiting slower melting or liquefaction at a given temperature (e.g., body temperature) and/or increased toughness. Liquefaction can involve the combined process of melting and hydration. The hydration properties of the implant material (e.g., a highly plasticized gelatin) may be affected by the hydrophilicity of the plasticizer, the hydrophilicity of the bioactive agents (e.g., if present in high concentrations), and degree of crosslinking. For example, greater crosslinking, for example increased dehydrothermal heat treatment, can result in stiffer (more resistant to deformation), tougher (e.g., less likely tear), and/or dryer materials; since hydration may be involved in the liquefaction process of a material, a decreased water content of the film or wrap can result in increased working times for the material, as the film may liquefy more slowly due to the decreased water content of the film or wrap material. Additional plasticizer may be included in the material, e.g., to reduce the stiffness with little or no increase in the degree of swelling of the material after insertion into a subject such as a human patient. In some embodiments and as shown in the below examples, a film, covering, or wrap of the present invention may have a working time of more than one hour.

The working times of an antimicrobial film of the present invention may be increased by lightly crosslinking the film. Crosslinking methods that may be used include, e.g., radiation, dehydrothermal heat treatment, and chemical crosslinking. Chemical crosslinking agents may be used to crosslink proteins using, e.g., carboxyl, carbonyl, sulfhydryl, amine or hydroxyl reactive agents. Homo bi (or poly) functional or hetero bi (or poly) functional agents can be used for crosslinking. In addition, enzymes can also be used for crosslinking. Common agents that may be used to promote crosslinking include, e.g., glutaraldehyde, di succinimide esters of N-hydroxy succinimide (NHS), such as polyethylene glycol NHS esters, carbo-diimide crosslinkers, maleimides, imidoesters, haloacetyls, pyridyl disulfides, hydrazides, glyoxals, sulfones, periodates, isocynates, ureas, disulfides. Activatable crosslinkers, such as photoactivated crosslinkers, can also be used including psoralens, aryl azides or diazirines. Radiation and dehydrothermal treatement may be preferably used in some embodiments, as they offer the benefit of not needing to introduce new chemical agents into the films.

Crosslinking of a film may in some embodiments preferably be performed prior to adding antimicrobial compound(s) to the film, since crosslinking can potentially adversely affect antimicrobial compound(s) in the film. For example, the heat associated with dehydrothermal crosslinking treatment can have undesirable impacts on the stability and residual activity of bioactive agents such as minocycline, rifampin, MeSNA, fatty acids or glycerol nitrates. Similarly, chemical crosslinking agents or radiation may react with bioactive agents. As shown in the below examples, different designs allow incorporation of bioactive agents into the films subsequent to partial crosslinking. For example, preformed pockets may be created in a film that allows for addition of bioactive agents, e.g., comprised in a formulation with a shorter working time such as, e.g., a gelatin formulation or a highly plasticized gelatin with a shorter working time, or in another liquid or solid formulation. As shown in the below examples, the working time and flexibility of films can be adjusted by the duration and temperature of dehydrothermal treatment, and the ductility can be affected by adjusting the quantity of plasticizer and/or water remaining in the film.

### XI. METHODS FOR REDUCING INFECTION AND SURGICAL COMPLICATIONS

In various aspects the antimicrobial bioabsorbable films of the present invention may be used to reduce or prevent infection or other complications, such as capsular contracture, that may be associated with the implantation of a medical device, such as a breast implant. In some embodiments, infections associated with breast reconstruction, breast implants, and/or breast tissue expanders may be reduced or substantially prevented. The bioabsorbable films may also be used to wrap a portion or all of an implanted device. The wrapping may occur before or during a surgery. In various embodiments, other complications of implanted devices may be reduced or substantially prevented such as, e.g., fibrosis, scaring, and/or formation of adhesions.

The films and wraps provided herein can be utilized in a variety of different surgeries. If desired, the films or wraps can be laminated on or applied to another implant or device such, e.g., a mesh or other structural devices or implant. In some embodiments the film or wrap (e.g., laminated on the device or implant or placed around an implant) is used to reduce or help prevent adhesion, infection, fibrosis, inflammation, or other procedural complication(s), and additional bioactive compounds can be included in the film or wrap to promote these effects (e.g., the bioactive compound may be an anti-inflammatory agent, and antimicrobial agent, etc.). The films and wraps provided herein can be applied to, laminated on, and/or used in surgical procedures with hernia meshes, pacemaker stabilizing envelopes, gynecologic meshes, neurologic/cranial overlays, spinal or nerve guides, tendon implants (e.g., a tendon implant used in a surgery of the hand, foot, shoulder, or knee, etc), periodontal implants, oral-maxilofacial implants, nerve stimulators, implantable pumps, ventricular assist devices, anastomotic couplers, pins, rods, screws, (such as surgical pins, rods, or screws used in an orthopedic or dental surgery), soft tissue pledgets or buttresses, wires, or cables. In some embodiments, the film or wrap is overlayed, layered on top of, or wrapped around at least a portion of a cartilage or orthopedic implant, or administered to a region of a cartilage or orthopedic surgery, to reduce or help prevent infection and/or other complication(s) following closure of the surgical site.

The following methods are provided as examples for how a wrap, covering, or film of the present invention may be applied to an implant in a surgical pocket. In some embodiments, an implant is fully wrapped with the substantially solid film prior to inserting it into a surgical pocket. Alternately, the wrap can be applied to the implant by lining all or part of the surgical pocket with the film and then inserting the implant into a subject, such as a human patient. In some cases, the bottom or certain portions of the surgical pocket can be lined with film and then additional film is draped over the top and sides of the implant prior to insertion.

Application of a wrap, film, or covering of the present invention can also be accomplished by converting a solid film of the present invention into a plurality of particles or smaller pieces (e.g., that are substantially solid at room temperature and that liquefy in situ at body temperature, like the solid film). The particles may be formed by cryomilling the solid film (e.g., a solid gelatin film) or by other mechanical (e.g., chopping, mincing, dicing) processes. Particles can also be directly formed from the molten gelatin material by dripping, dispersing droplets or emulsifying in a non-solvent, such as an oil or silicone fluid, and then cooling to solidify. Particles can further be formed by extruding a molten gelatin into thin filaments that are chopped upon cooling. Particles can be directly molded by extruding the molten gelatin into molds with particle shapes or indentations and then cooling. Particles can be directly applied to the implant or in the surgical pocket prior to placement of an implant. Particles can also be suspended in a volatile non-solvent propellant and then sprayed. Examples of volatile non-solvent propellants are butane, propane, volatile dimethicones and cyclomethicones and hydrofluoroalkanes such as tetrafluoroethane, difluoroethane and hexafluoropropane. Particles can also be suspended in fluids that are absorbable, drain, or evaporate and spread in the surgical pocket or on the implant. Plasticizing agents such as aliphatic polyols, sugars, polyethylene glycols and glycerols, aqueous fluids and short chain or unsaturated lipids can be used to facilitate spreading. A plasticizing agent may be used instead of or in combination with a volatile non-solvent propellant.

### XI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Tackiness of Minocycline+Rifampin Loaded Wrap Formed

An approximately 1 mm thick laminate wrap was formed by first producing an aqueous solution of highly plasticized porcine gelatin containing 40 g gelatin and 48 glycerol. The 80° C. solution was poured into a tray and dehydrated by heating at 175 F for 24 hr and then dehydrothermally crosslinked by raising the temperature to 225 F for an additional 2 hrs. A top layer was then cast on top of the crosslinked layer by preparing a hot solution containing 37 g gelatin and 37 g glycerol. As the solution cooled a solution of Minocycline and Rifampin dissolved in ethanol was mixed in when the temperature dropped below 50° C. The warn solution containing gelatin, glycerol, Minocycline and Rifampin was poured/cast on top of the crosslined layer and allowed to cool. The antibiotics uniformly diffused through the laminate wrap. The cooled laminate was allowed to dry an additional 48 hours under dry air convection at 20° C. The wrap was then removed from its tray placed between parchment paper, wrapped in aluminum foil then vacuum sealed in plastic. After 2 weeks storage at 20° C. the wrap was removed from its packaging and the parchment paper liner examined. Significant orange color had transferred to the liner from both surfaces as shown in the photograph below. The wrap had tacky adhesion to the liner but could be peeled off without tearing it using moderate force. Results are shown in FIG. 1.

### Example 2

### Formation of Blank Wrap

An approximately 1 mm thick laminate wrap was formed by first producing an aqueous solution of highly plasticized porcine gelatin containing 40 g gelatin and 48 glycerol. The 80° C. solution was poured into a tray and dehydrated by heating at 175 F for 24 hr and then dehydrothermally crosslinked by raising the temperature to 225° F. for an additional 2 hrs. A top layer was then cast on top of the crosslinked layer by preparing and casting over it a hot solution of 37 g gelatin and 37 g glycerol. The cooled laminate was allowed to dry an additional 48 hours under dry air convection at 25° C.

### Example 3

Impregnation of a Blank Wrap with Minocycline and Rifampin by Spraying at 20° C.

100 mg of Rifampin was dissolved in 1.5 mL ethanol at 20° C. This was added to 12 mL water at 20° C. 100 mg Minocycline was dissolved in the solution at 20° C. 6.75 mL of the solution was uniformly sprayed at 20° C. using a spray gun over the surface of the blank wrap formed in Example 2. This was allowed 1 hr to absorb into the bank wrap at 20° C. The wrap was then flipped onto its other side and 6.75 mL of the solution was uniformly sprayed at 20° C. using a spray gun over the surface of the wrap. This was allowed 1 hr to absorb into the bank wrap at 20° C.

### Example 4

### Cryoprocessing of Impregnated Wrap

The impregnated wrap formed in Example 3 was sealed in clear plastic and frozen at 0 F for 24 hr. The wrap became firmer but could still be bent or flexed without it cracking. The impregnated drugs remained uniformly dispersed in the wrap. The cryoprocessed wrap (still sealed in plastic) was allowed to warm to room temperature where it essentially returned to the same physical form as before cryoprocessing.

### Example 5

### Sublimating Cryoprocessed Impregnated Wrap

A cryoprocessed impregnated wrap from Example 4 was removed from its plastic packaging and allowed to sublimate for 10 days at 0 F under dry air convection. Upon completion of sublimation cycle but still at 0 F the wrap remained ductile and could be easily flexed without cracking. Upon warming to 20 C the wrap was noticeable less tacky than the wrap from example 1 yet retained similar color intensity of the Minocycline and Rifampin. This wrap was then placed between parchment paper, wrapped in aluminum foil then vacuum sealed in plastic. After 2 weeks storage at 25 C the wrap was removed from its packaging and the parchment paper liner examined. No visible color transfer of Minocycline and Rifampin to the liner was observed.

### Example 6

### Antimicrobial Activity of Wraps

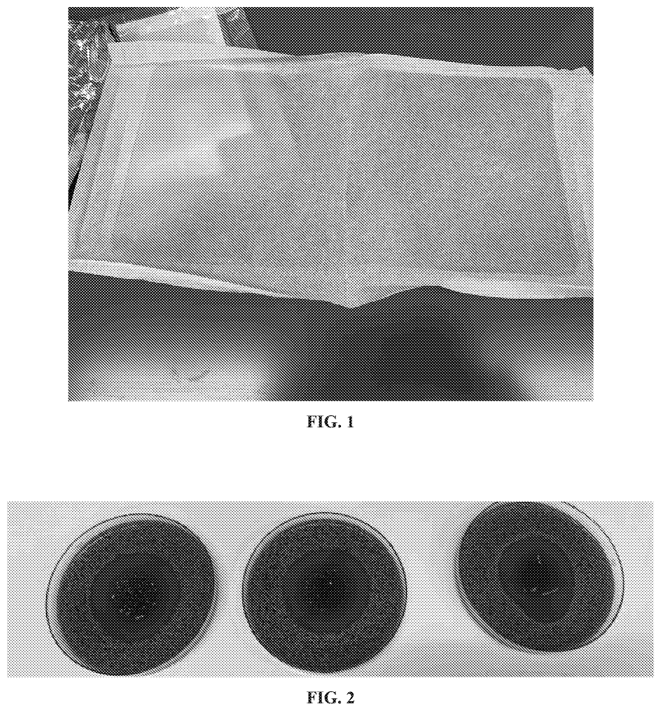

Antimicrobial activity of wraps formed in Examples 1, 3 and 5. Zones of Inhibition against a clinical isolate of Methicillin Resistant Staphylococcus aureus (MRSA) were measured for 1 cm wrap disks from Examples 1, 3 and 5 using the Kirby-Bauer method. Sheeps blood aga plates were streaked with inocula of MRSA in Mueller Hinton Broth. A 1 cm disk of wrap was placed in the center of the plate and then the plate was incubated covered for 24 hrs at 37 C. MRSA colonized the plate in areas where diffusion of antibiotics was at concentrations below the minimum inhibitory concentration. Above the minimum inhibitory concentration in the region near the disks the MRSA failed to grow. The photograph below arranged in order (left to right) Example 1, Example 3 and Example 5 produced zones of inhibition of approximately 23 mm indicating the antibiotic activity was preserved during cryoprocessing and that antimicrobial activity of the cryoprocessed wrap was not impaired by sublimation. Results are shown in FIG. 2.

### Example 7

### Vacuum Sublimating Cryoprocessed Wrap

A Minocycline+Rifampin impregnated at 20° C. was prepared as in Example 3. The wrap was placed in a freeze dryer and cryoprocessed at approximately −20° C. and sublimated by exposure to very low environmental pressure produced by a vacuum pump. The cryoprocessed sublimated wrap was removed after 24 hrs and had surface properties similar to the cryoprocessed sublimated wrap produced in example 5. The wrap produced by the method in this example showed that effective sublimation could be accelerated by application of vacuum.

### Example 8

### pH Adjusted Vacuum Sublimated Cryoprocessed Wrap

A blank wrap was produced by the method in Example 2. The impregnation solution was prepared as in example 3 except the pH was measured prior to spraying. It was 3.93. The pH was adjusted to 7.2 by addition of base (sodium hydroxide) and then impregnated at 20° C. as in Example 3. This wrap was then sublimated and cryoprocessed as in Example 5. Similar surface properties were obtained as with the wrap in Example 5.

### Example 9

### pH Adjusted Wrap Using Different Base Solutions

Neutral pH wraps were prepared as in Example 8 except ammonium hydroxide was used in one instance to adjust the pH to 7.2, sodium carbonate was used in another instance and tetrasodium EDTA was used in another instance.

### Example 10

### Ascorbic Acid Impregnated Vacuum Sublimated Cryoprocessed Wrap Containing Minocycline and Rifampin (M+R)

Ascorbic acid impregnated vacuum sublimated cryoprocessed M+R wrap were generated as follows. Wrap was prepared as in Example 8 except 100 mg ascorbic acid (antioxidant) was added to the impregnation solution prior to pH adjustment. Cryoprocessing and sublimation was performed as in Example 7.

### Example 11

### Magnesium Sulfate Impregnated Vacuum Sublimated Cryoprocessed Wrap

Magnesium Sulfate impregnated vacuum sublimated cryoprocessed wrap—A wrap was prepared as in Example 10 except 269 mg Magnesium sulfate was added to the impregnation solution.

### Example 12

### Laminating Vacuum Sublimated Cryoprocessed Wraps

Laminating vacuum sublimated cryoprocessed wraps (M layer and R layer-wet and apply pressure) were generated as follows. An M+R wrap was prepared by first forming one wrap containing only M and one wrap containing only R. The M wrap was formed as in Example 8 except only Minocycline was added to the Wrap impregnation solution (not Rifampin). The R wrap was formed as in Example 8 except on Rifampin was added to the wrap impregnation solution. The two wraps were laminated at 20 C by applying a minute layer of cooled but not-yet-gelled gelatin solution (1 g gelatin in 10 mL water, heated to 80 C to dissolve the gelatin then allowed to cool but applied prior to gelation) to the cast side of one of the wraps to generate surface tackiness and then overlaying the crosslinked side of the other wrap and applying strong compressive pressure to laminate. The Laminated wraps were vacuum dried prior to sealing.

### Example 13

### Laminating Vacuum Sublimated MR and MesNA Wraps

Laminating vacuum sublimated MR and MesNA wraps were generated as follows. One MR wrap was prepared as in example 12 and a second wrap impregnated with 2-Mercaptoethane sulfonic acid (MeSNA) but no M or R was prepared as in Example 12 except 1 g sodium 2-mercaptoethanesulfonate (MeSNA) was added to the impregnation solution instead of M and R. The wraps were then laminated together as in Example 12.

### Example 14

### Wrap Formed on 30 Degree Contact Angle Surface

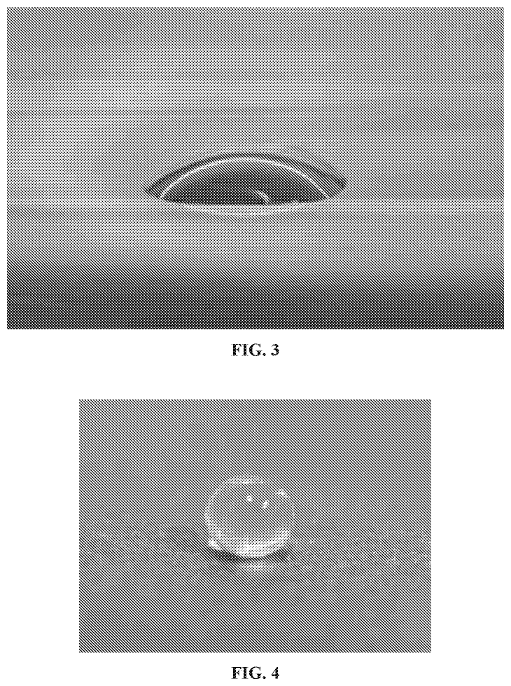

The blank wrap of Example 2 was formed on a glass surface. The water contact angle on the surface was measured by applying a single drop of deionized water to the surface of the pan and capturing a photograph under magnification (shown below). The contact angle was measured as the angle formed between the droplet and the horizontal plane (in this case approximately 30 degrees). This spreading of the drop is characteristic of hydrophilic surfaces. The wrap formed adhered strongly to the surface, was difficult to peel off and tore in places when separating from the glass pan surface. This demonstrated that surfaces with excessive adhesion are able to form uniform wraps but the adhesive strength can be greater than the tensile strength of the wrap so it was not possible to separate it from the surface for impregnation without damaging it. Results are shown in FIG. 3

### Example 15

Wrap with Surface Coating and 160 Degree Contact Angle

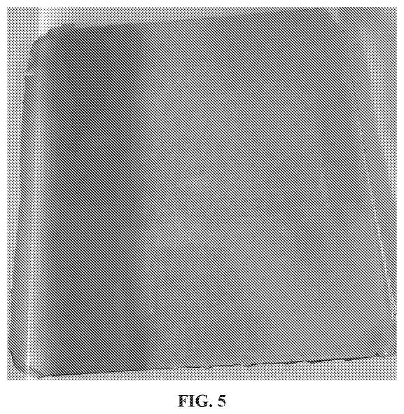

A blank wrap was formed as in Example 14 except on a pan with Neverwet Superhydrophobic surface coating (Ross Nanotechnology, Leola, PA). The contact angle was measured as approximately 160 degrees and has characteristic beading into a sphere from contact with a highly hydrophobic surface. A wrap of consistent thickness failed to form because it contracted away from the edges of the pan as it cured. The wrap also formed blisters during curing. This demonstrated that a surface with too little adhesion to the cured blank wrap is not able to maintain dimensional integrity during curing and though easy to remove is not suitable for impregnation and further use. Results are shown in FIG. 4.

### Example 16

### Blank Wrap Formed on 75 Degree Contact Angle Surface

A blank wrap was formed as in Example 14 on a ceramic coated pan. The water contact angle was measure as 75 degrees. The wrap formed with this pan retained dimensional integrity and was able to be separated (peeled away) from the surface with moderate force and without damaging (tearing) it. This blank wrap was well suited for impregnation and further processing.

### Example 17

### Blank Wrap Formed on 90 Degree Contact Angle Surface

A blank wrap was formed as in Example 14 on a stainless-steel pan. The water contact angle was measure as 90 degrees. The wrap formed with this pan retained dimensional integrity and was able to be separated (peeled away) from the surface without damaging (tearing) it. This blank wrap was well suited for impregnation and further processing.

### Example 18

### Blank Wrap Formed on 130 Degree Contact Angle Surface

A blank wrap was formed as in Example 14 on a porous PTFE lined pan. The water contact angle was measure as 130 degrees. The wrap formed with this surface retracted and curled at the edges but retained dimensional integrity in its middle. It was easy to separate (peel away) from the surface without damaging (tearing) it. The curled edges of this wrap could be trimmed and the remaining middle portion was suitable for impregnation and further processing.

### Example 19

### Spatially Separating Drugs on Spray Coating Wraps

A blank wrap prepared as in Example 2 was spray impregnated with Minocycline and Rifampin as in Example 3 except only Minocycline was sprayed on one side of the wrap and only Rifampin was sprayed the other side. The two spray solutions were prepared as described in example 12. Upon drying the drugs remained separated on different sides of the wrap as illustrated in the photograph below (Minocycline is yellow [right] side and Rifampin is orange [left] side). Note the clear unimpregnated center strip between the two impregnated regions showing that the two drugs remained separated in the wrap. Results are shown in FIG. 5.

### Example 20

### Packaging Wraps Using Different Liners

An MR Wrap prepared as in Example 7 was cut into thirds. One third was inserted in between parchment paper, ⅓ in between silicone coated parchment paper and ⅓ in between PTFE thin sheeting. The 3 samples were stored as in Example 1. The parchment paper had feint traces of transferred antibiotic (orange color) while the silicone and PTFE liners had none.

### Example 21

Impregnation of Wrap with Multiple Antimicrobial Agents at Low pH Followed by In Situ Adjustment of Wrap pH

A wrap was prepared as described in Example 8, with the modification that the pH of the solution was adjusted to 2 by addition of HCl prior to dissolving Rifampin. Minocycline was subsequently dissolved in the pH 2 solution. Wrap was spray-impregnated at pH 2 and allowed to absorb all liquid. NaOH was then spray impregnated to adjust the wrap pH to 4. For the volumes in Example 3, about 500 microliters of NaOH was required to be spray impregnated into the wrap to perform the pH adjustment. Wrap was freeze dried under vacuum prior to storage.

### Example 22

Impregnation of Wrap with Multiple Antimicrobial Agents at Low pH Followed by In Situ Adjustment of Wrap pH

A wrap was prepared as described in Example 8, with the modification that the pH of the solution was adjusted to 2 by addition of acetic acid prior to dissolving Rifampin. Minocycline was subsequently dissolved in the pH 2 solution. Wrap was spray-impregnated at pH 2 and allowed to absorb all liquid. NaOH was then spray impregnated to adjust the wrap pH to 4. For the volumes in Example 3, about 500 microliters of NaOH was required to be spray impregnated into the wrap to perform the pH adjustment. Wrap was freeze dried under vacuum prior to storage.

### Example 23

Impregnation of Wrap with Multiple Antimicrobial Agents at Low pH without In Situ Adjustment of Wrap pH

A wrap was prepared as described in Example 8, with the modification that the pH of the solution was adjusted to 2.7 by addition of HCl prior to dissolving Rifampin. Minocycline was subsequently dissolved in the pH 2.7 solution. Wrap was spray-impregnated at pH 2 and allowed to absorb all liquid. Wrap was then freeze dried under vacuum. The freeze dried wrap was immersed in an excess of deionized water and allowed to swell. The pH of the water was measured until it stabilized. The final pH was about 4.5.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Publication No. 20080241212
U.S. Publication No. 20080128315
U.S. Publication No. 20110082545
U.S. Publication No. 20110082546
U.S. Publication No. 20120052292
U.S. Publication No. 20120123535
U.S. Pat. No. 3,042,524
U.S. Pat. No. 5,622,740
U.S. Pat. No. 10,953,137
Kang et al., Biomaterials; 20(14):1339-44, July 1999.
Pittet et al., Infection in breast implants. Lancet Infect Dis.;5(2):94-106, February 2005.
Rosenblatt et al., BioMed research international; Nov. 7; 2017.
Viola et al, Infection Control and Hospital Epidemiology; 35(1):75-81, 2014.
Van Vlierberghe et al, Biomacromolecules;8(2):331-7, February 2007.
Van Vlierberghe et al, Journal of Biomaterials Science, Polymer Edition; 20(10):1417-38, January 2009.
Zaoui-Djelloul-Daouadji et al, The Journal of Chemical Thermodynamics; 69:165-71, February 2014.

1. A biodegradable covering for a medical implant, the covering comprising a highly plasticized gelatin and at least one drug to reduce infection or capsular contraction,
wherein the highly plasticized gelatin consists essentially of gelatin and from about 35% to about 60% plasticizer,
wherein the plasticized gelatin has a melting temperature of less than 38° C., and wherein the biodegradable covering has been subjected to cryoprocessing or freeze-drying.

2. The covering of claim 1, wherein the cryoprocessing or freeze-drying comprises cooling the temperature to from about 0° C. to about −40° C.

3. The covering of claim 1, wherein the cryoprocessing or freeze-drying comprises cooling the temperature to from about −10° C. to about −25° C.

4. The covering of claim 1, wherein the cryoprocessing or freeze-drying comprises cooling the temperature to from about −10° C. to about −20° C.

5. The covering of any one of claims 1-4, wherein the cryoprocessing comprises dry air convection or applying dry air to the covering.

6. The covering of any one of claims 1-5, wherein the cryoprocessing or freeze-drying occurs for from 1 minute to 2 weeks, more preferably from about 1 hour to about 2 weeks.

7. The covering of claim 6, wherein the cryoprocessing occurs for about 1-24 hours.

8. The covering of any one of claims 1-4, wherein the freeze drying comprises applying reduced atmospheric pressure to the covering.

9. The covering of claim 8, wherein the reduced atmospheric pressure results from a vacuum pump.

10. The covering of claim 8, wherein the freeze-drying occurs for about 1-24 hours.

11. The covering of claim 10, wherein the freeze-drying occurs for about 1-8 hours.

12. The covering of claim 1, wherein the plasticized gelatin has a melting temperature of 27-37° C.

13. The covering of claim 1, wherein the plasticized gelatin has a melting temperature of 30-37° C.

14. The covering of claim 12, wherein the plasticized gelatin comprises about 40-60% plasticizer.

15. The covering of claim 14, wherein the plasticizer is glycerol, a propylene glycol, a sugar, a carbohydrate, an amino acid, a salt, an acid, or a polyol.

16. The covering of claim 15 wherein the plasticizer is glycerol.

17. The covering of claim 1, wherein at least a portion of an inner surface of the covering is substantially sticky or adhesive, and a portion of or substantially all of an outer surface of the covering is substantially lubricious.

18. The covering of claim 1, wherein at least a portion of a surface of the covering has been treated with a gluconic acid solution.

19. The covering of claim 1, wherein at least a portion of a surface of the covering has been treated with a glycerol-gelatin liquid comprising about 60-90% glycerol or a solution comprising a carbohydrate, a starch, or a sugar.

20. The covering of claim 1, wherein the covering is sufficient in size or shaped to cover a breast implant.

21. The covering of claim 20, wherein the covering is shaped as a film, a wrap, a pouch or a bag.

22. The covering of claim 21, wherein the covering is a pouch or a bag; wherein the covering has a central region and a plurality of lateral appendages, or the covering is substantially star-shaped.

23. The covering of claim 1, wherein the covering comprises a plurality of biodegradable layers.

24. The covering of claim 1, wherein the at least one drug is selected from the group consisting of an antimicrobial agent, an anti-inflammatory agent, an anti-scarring agent, a hemostatic agent, an anti-neoplastic agent, a calcium channel blocker, a leukotriene inhibitor, an antifibrotic agent, a fibrotic agent, an anesthetic, an analgesic, and thrombolytic agent.

25. The covering of claim 1, wherein the at least one drug is comprised in a fiber, a bead, a particle, a liposome, a microsphere, or a nanosphere.

26. The covering of claim 24, wherein the at least one drug is an antimicrobial agent.

27. The covering of claim 26, wherein the antimicrobial agent is bacitracin, cephalexin, gentamicin, an antiseptic, a chelator, chlorhexidine, gendine, gardine or mixtures thereof.

28. The covering of claim 27, wherein the antiseptic is hydrogen peroxide, chlorhexidine, gendine or gardine.

29. The covering of claim 27, wherein the covering further comprises mercaptoethane sulfonate (MeSNA), minocycline, rifampin, and/or glyceryl trinitrate (GTN).

30. The covering of claim 26, wherein the covering further comprises nitroglycerin or a nitric oxide donor.

31. The covering of claim 26, wherein the at least one drug is a leukotriene inhibitor.

32. The covering of claim 31, wherein the leukotriene inhibitor is a leukotriene receptor antagonist selected from the group consisting of acitazanolast, iralukast, montelukast, pranlukast, verlukast, zafirlukast, and zileuton.

33. The covering of any one of claims 1-32, wherein the covering comprises one, two, three, or all of mercaptoethane sulfonate (MeSNA), minocycline, rifampin, or glyceryl trinitrate (GTN).

34. The covering of claim 26, wherein the antimicrobial agent is minocycline.

35. The covering of claim 34, wherein the covering comprises minocycline and rifampin.

36. The covering of any one of claims 34-35, wherein the covering has a pH of about 6-8.

37. The covering of claim 36, wherein the covering has a pH of about 7-7.4.

38. The covering of claim 37, wherein the covering comprises minocycline, rifampin, and mercaptoethane sulfonate.

39. The covering of any one of claims 33-38, wherein the covering further comprises glyceryl trinitrate (GTN).

40. The covering of any one of claims 33-39, wherein the covering further comprises a fatty acid or monoglyceride.

41. The covering of claim 40, wherein the fatty acid is a C₆₋₁₂ alkanoic acid.

42. The covering of claim 41, wherein the fatty acid is a C₆₋₁₀ alkanoic acid.

43. The covering of claim 41, wherein the fatty acid is hexanoic acid, octanoic acid, decanoic acid, dodecanoic acid, caprylic acid (octanoic acid), caproic acid, or lauric acid.

44. The covering of claim 43, wherein the fatty acid is caprylic acid (octanoic acid).

45. The covering of claim 33, wherein the covering comprises glyceryl trinitrate (GTN) and capyrilic acid.

46. The covering of any one of claims 1-45, wherein at least a portion of the covering has been exposed to crosslinking.

47. The covering of claim 46, wherein at least half of the covering has been exposed to crosslinking.

48. The covering of claim 46, wherein the crosslinking comprises exposing at least a portion of the covering to radiation.

49. The covering of claim 46, wherein the crosslinking comprises exposing at least a portion of the covering to a dehydrothermal heat treatment.

50. The covering of claim 46, wherein the crosslinking is further defined as mild or partial crosslinking.

51. The covering of claim 46, wherein the crosslinking is sufficient to increase the working time, toughness, or stiffness of the covering.

52. The covering of claim 46, wherein said portion comprises an antimicrobial agent.

53. The covering of claim 52, wherein the antimicrobial agent is minocycline, rifampin, chlorhexidine, gendine, or gardine.

54. The covering of claim 46, wherein said portion comprises minocycline and rifampin.

55. The covering of claim 46, wherein said portion further comprises mercaptoethane sulfonate (MeSNA), glyceryl trinitrate (GTN), or a C₆₋₁₀ alkanoic acid.

56. The covering of claim 55, wherein the a C₆₋₁₀ alkanoic acid is caprylic acid.

57. The covering of any one of claims 46-56, wherein the covering comprises regions that have been exposed to crosslinking and regions that have not been exposed to crosslinking.

58. The covering of claim 57, wherein the regions that have not been exposed to crosslinking comprise the drug, and wherein the regions that have been exposed to crosslinking do not comprise the drug.

59. The covering of claim 57, wherein both the regions that have not been exposed to crosslinking and the regions that have been exposed to crosslinking both comprise the drug.

60. The covering of claim 57, wherein the regions that have not been exposed to crosslinking comprise the drug, and wherein the regions that have been exposed to crosslinking do not comprise the drug.

61. The covering of claim 58, wherein the regions that have not been exposed to crosslinking comprise minocycline and rifampin.

62. The covering of claim 61, wherein the regions that have not been exposed to crosslinking further comprise glyceryl trinitrate (GTN), mercaptoethane sulfonate (MeSNA), or caprylic acid.

63. The covering of any one of claims 1-45, wherein at least a portion of the covering has not been exposed to crosslinking.

64. The covering of any one of claims 1-63, wherein the covering comprises or consists of a single layer.

65. The covering of claim 64, wherein the covering comprises regions that have been exposed to crosslinking and regions that have not been exposed to crosslinking.

66. The covering of claim 65, wherein the drug is comprised in the regions that have not been exposed to crosslinking.

67. The covering of claim 65, wherein the drug is comprised in the regions that have been exposed to crosslinking.

68. The covering of any one of claims 65-66, wherein the regions that have not been exposed to crosslinking are present in the covering in a pattern of shapes or in a sponge-like pattern.

69. The covering of claim 68, wherein the shapes comprise a plurality of substantially circular or oval shapes.

70. The covering of any one of claims 1-63, wherein the covering has multiple layers.

71. The covering of claim 70, wherein the covering has at least 2 layers.

72. The covering of claim 71, wherein the covering has 2 layers.

73. The covering of any one of claims 70-73, wherein a layer has been exposed to crosslinking.

74. The covering of claim 73, wherein the layer comprises an antimicrobial agent.

75. The covering of claim 74, wherein the layer has been exposed to a dehydrothermal heat treatment and subsequently contacted with a solution containing the antimicrobial agent.

76. The covering of claim 75, wherein the layer is dried or exposed to a dehydrothermal heat treatment after being contacted with said solution.

77. The covering of any one of claims 75-76, wherein said solution comprises an alcohol and water.

78. The covering of claim 77, wherein the alcohol is ethanol or methanol.

79. The covering of any one of claims 77-78, wherein the alcohol comprises about 1-50% (v/v) of the solution.

80. The covering of any one of claims 72-76, wherein the solution comprises gelatin and glycerol.

81. The covering of claim 72, wherein the covering comprises a first layer comprising a partially crosslinked plasticized gelatin and a second layer comprising a plasticized gelatin that has not been crosslinked, wherein the second layer comprises the drug.

82. The covering of claim 81, wherein the second layer comprises minocycline and rifampin.

83. The covering of claim 70, wherein the highly plasticized gelatin is comprised in an inner layer or a middle layer of the covering.

84. The covering of claim 83, wherein an outer layer of the covering has a melting temperature of greater than 38° C.

85. The covering of claim 70, wherein the covering has 3, 4, 5, or 6 layers.

86. The covering of claim 85, wherein the covering has 3 layers, wherein the 3 layers are an outer layer, a middle layer, and an inner layer.

87. The covering of claim 86, wherein the outer layer, the inner layer, or the middle layer of the covering comprises the drug.

88. The covering of claim 86, wherein the middle layer comprises the highly plasticized gelatin.

89. The covering of claim 88, wherein the inner layer and/or the outer layer has a melting temperature of greater than 38° C.

90. The covering of claim 87, wherein the outer layer and inner layer have been exposed to crosslinking.

91. The covering of claim 87, wherein regions of the middle layer have been exposed to crosslinking and regions of the middle layer have not been exposed to crosslinking, wherein said at least one drug is comprised in a least some of the regions that have not been exposed to crosslinking.

92. The covering of any one of claims 70-91, wherein one or all of the edges of the covering are melted or welded together.

93. The covering of claim 92, wherein the covering comprises at least three layers, and wherein the edges of the outermost layers have been melted or welded together by the application of heat.

94. The covering of claim 93, wherein the outermost layers are partially crosslinked, and wherein an inner layer comprises the highly plasticized gelatin and the drug.

95. The covering of claim 94, wherein the inner layer comprises minocycline and rifampin.

96. The covering of claim 94, wherein said application of heat is via heat gun, food sealer, or laser.

97. The covering of any one of claims 1-91, wherein the drug is an antimicrobial agent, and wherein the covering comprises a second drug.

98. The covering of claim 97, wherein the second drug is an anti-inflammatory agent, an anti-scarring agent, a hemostatic agent, an anti-neoplastic agent, a calcium channel blocker, a leukotriene inhibitor, a leukotriene inhibitor, an antifibrotic agent, a fibrotic agent, an anesthetic, an analgesic, or a thrombolytic agent.

99. The covering of any one of claims 97-98, wherein the covering comprises minocycline and rifampin.

100. The covering of claim 99, wherein the covering further comprises glyceryl trinitrate (GTN), mercaptoethane sulfonate (MeSNA), caprylic acid or tranilast.

101. The covering of any one of claims 97-100, wherein the antimicrobial agent and the second drug are comprised in overlapping regions of the covering.

102. The covering of claim 101, wherein the antimicrobial agent and the second drug are comprised on a surface of the covering.

103. The covering of claim 101, wherein the antimicrobial agent and the second drug are comprised or dispersed within the covering.

104. The covering of any one of claims 97-100, wherein the antimicrobial agent and the second drug are comprised in non-overlapping regions of the covering.

105. The covering of claim, wherein the antimicrobial agent and the second drug are comprised on opposite sides of the covering.

106. The covering of any one of claims 1-100, wherein the highly plasticized gelatin is comprised on an adhesive backing.

107. The covering of claim 106, wherein the adhesive backing is translucent.

108. The covering of claim 106 or 107, wherein the adhesive backing is part of a bandage or wound dressing.

109. The covering of claim 108, wherein the highly plasticized gelatin is translucent, and wherein bandage or wound dressing allows for viewing of skin or tissue under the bandage or wound dressing.

110. The covering of any one of claims 1-109, wherein the covering is comprised on a backing.

111. The covering of claim 110, wherein the backing comprises silicone, a silicone coating, or PTFE.

112. The covering of claim 111, wherein the backing is further defined as a storage backing or a backing that can be removed prior use.

113. The covering of any one of claims 1-112, wherein the pH of the covering is about 6-8.

114. The covering of 113, wherein the pH of the covering is about 7-7.4.

115. The covering of any one of claims 1-112, wherein the pH of the covering is from about 1 to about 7.

116. The covering of claim 115, wherein the pH of the covering is from about 1 to about 4.

117. The covering of claim 115, wherein the pH of the covering is from about 2 to about 4 or about 2.25-2.75.

118. The covering of claim 115, wherein the pH of the covering is from about 4 to about 7.

119. The covering of claim 118, wherein the pH of the covering is from about 4 to about 6.

120. The covering of any one of claims 115-119, wherein the covering comprises rifampin and a tetracycline.

121. The covering of claim 120, wherein the tetracycline is minocycline.

122. The covering of any one of claims 1-112, wherein the pH of the covering is from about 8 to about 12.

123. The covering of any one of claims 115-122, wherein the covering has been substantially dehydrated or freeze-dried.

124. A kit comprising a medical implant and the biodegradable covering of any one of claims 1-123.

125. The kit of claim 124, wherein the medical implant is a breast implant, a hernia mesh, a pacemaker stabilizing envelope, a gynecologic mesh, a neurologic or cranial overlay, a nerve guide, a tendon surgery implant, a periodontal implant, an oral-maxilofacial implant, nerve stimulator, implantable pump, ventricular assist device, anastomotic coupler, pin, rod, screw, soft tissue pledget or buttress, wire, or cable.

126. The kit of claim 125, wherein the nerve guide is a spinal nerve guide.

127. The kit of claim 125, wherein the tendon surgery implant is configured for use in a tendon surgery of the hand, foot, shoulder, or knee.

128. The kit of any one of claims 124-127, wherein the biodegradable covering is freeze dried or dehydrated, and wherein the biodegradable covering is comprised in a container means comprising a moisture barrier material.

129. The kit of claim 128, wherein the moisture barrier material is aluminum foil, plastic, or glass.

130. The kit of any one of claims 128-129, wherein the container means comprises a release lining film.

131. The kit of claim 130, wherein the release lining film is adjacent to or in physical contact with the biodegradable covering.

132. The kit of any one of claims 130-131, wherein the release lining film is a paper liner, a silicone liner, or a polytetrafluoroethylene (PTFE) liner.

133. The kit of claim 132, wherein the paper liner comprises a silicone coating or a fluoropolymer coating.

134. The kit of any one of claims 128-133, wherein the biodegradable covering has been sealed in the container means in (i) a substantially anhydrous environment and/or (ii) in a reduced oxygen or oxygen-free atmosphere.

135. The kit of any one of claims 124-134, wherein the kit comprises an oxygen absorbing packet.

136. The kit of claim 135, wherein the oxygen absorbing packet comprises iron powder.

137. The kit of any one of claims 124-136, wherein the kit comprises a moisture absorbing packet.

138. The kit of claim 137, wherein the moisture absorbing packet comprises a silica gel or an epoxy resin.

139. The kit of any one of claims 124-138, wherein the biodegradable covering has been sterilized by exposure to electromagnetic radiation.

140. The kit of claim 139, wherein the electromagnetic radiation comprises gamma radiation or E-beam radiation.

141. A medical implant assembly comprising a biodegradable covering of claim any one of claims 1-123 and a medical implant.

142. The medical implant assembly of claim 141, wherein the medical implant is a breast implant, a hernia mesh, a pacemaker stabilizing envelope, a gynecologic mesh, a neurologic or cranial overlay, a nerve guide, a tendon surgery implant, a periodontal implant, an oral-maxilofacial implant, nerve stimulator, implantable pump, ventricular assist device, anastomotic coupler, pin, rod, screw, soft tissue pledget or buttress, wire, or cable.

143. The medical implant assembly of claim 142, wherein the nerve guide is a spinal nerve guide.

144. The medical implant assembly of claim 142, wherein the tendon surgery implant is configured for use in a tendon surgery of the hand, foot, shoulder, or knee.

145. The medical implant assembly of claim 141, wherein the medical implant is a breast implant.

146. A method for reducing at least one post-surgical indication from breast augmentation or breast reconstruction in a subject, the method comprising surgically implanting into the subject the breast implant assembly of claim 145.

147. The method of claim 146, wherein the biodegradable covering is a film, and wherein the method comprises wrapping the breast implant with the biodegradable covering prior to insertion.

148. The method of claim 147, the method further comprising trimming excess film prior to said implanting.

149. The method of claim 147, wherein said wrapping occurs prior to a surgery for said implantation.

150. The method of claim 147, wherein said wrapping occurs during a surgery that comprises said implantation.

151. The method of claim 146, wherein the indication is selected from the group consisting of infection, inflammation, capsular contracture, adhesion, and scarring.

152. The method of claim 146, wherein the biodegradable covering is used to line or cover part or all of a region in the subjects body, wherein the breast implant is subsequently placed on the biodegradable covering, and wherein the covering is subsequently used to cover the breast implant.

153. A transcutaneous device assembly comprising a biodegradable covering of claim any one of claims 1-123 that is wrapped around at least a portion of the transcutaneous device.

154. The assembly of claim 153, wherein the transcutaneous device is an electrical nerve stimulation device, a catheter, a screw, a rod, a pin, a wire, a collar, a tube, a surgical drain, a hernia mesh, a pacemaker stabilizing envelope, a gynecologic mesh, a neurologic or cranial overlay, a nerve guide, a tendon surgery implant, a periodontal implant, an oral-maxilofacial implant, nerve stimulator, implantable pump, ventricular assist device, anastomotic coupler, pin, rod, screw, soft tissue pledget or buttress, wire, or cable.

155. The assembly of claim 154, wherein the transcutaneous device is a surgical drain.

156. A method for reducing at least one post-surgical indication from implantation of a transcutaneous device in a subject, the method comprising surgically implanting into the subject the transcutaneous device assembly of claim 153.

157. The method of claim 156, wherein the subject is a human patient.

158. The method of any one of claims 156-157, wherein the portion of the transcutaneous device that is placed in the subject is covered by said covering.

159. The method of claim 158, wherein the transcutaneous device is secured outside of the body of the subject with a wound dressing or bandage.

160. The method of any one of claims 156-159, wherein the biodegradable covering has a pH of less than about 4, about 1-3, about 1-2.5, about 2-4, or about 1, 1.5, 2, 2.5, 2.6, 2.7, 2.75, 2.8, 3, 3.5, 4, or any range derivable therein.

161. The method of claim 160, wherein the biodegradable covering has a pH of from about 1.5 to about 2.5.

162. The method of any one of claims 156-159, wherein the biodegradable covering has a pH of less than about 4-7, about 4-6, or about 4, 4.5, 5, 5.5, 6, 6.5, 7, or any range derivable therein.

163. The method of any one of claims 160-162, wherein the pH of the biodegradable covering is raised to a pH of at least about 4 in situ by application of an alkaline solution to the biodegradable covering.

164. The method of claim 163, wherein the biodegradable covering has been dehydrated or freeze dried prior to application of the alkaline solution.

165. The method of claim 164, wherein the in situ application is performed just prior to the surgically implanting into the subject.

166. The method of claim 165, wherein the alkaline solution has a pH of at least about 10, 11, 12, or any range derivable therein.

167. The method of any one of claims 164-166, wherein the alkaline solution comprises a second drug.

168. The method of claim 167, wherein the second drug is an antimicrobial agent, an anti-inflammatory agent, an anti-scarring agent, a hemostatic agent, an anti-neoplastic agent, a calcium channel blocker, a leukotriene inhibitor, an antifibrotic agent, a fibrotic agent, an anesthetic, an analgesic, or a thrombolytic agent.

169. The method of any one of claim 156-159, wherein the biodegradable covering has a pH of from about 8 to about 12.

170. The method of any one of claim 169, wherein the pH of the biodegradable covering is lowered to a pH of less than about 10 in situ by application of an acidic solution to the biodegradable covering.

171. The method of claim 170, wherein the biodegradable covering has been dehydrated or freeze dried prior to application of the acidic solution.

172. The method of claim 171, wherein the acidic solution has a pH of about 1-4, about 4-7, or about 4-6.

173. The method of claim 172, wherein the in-situ application is performed prior to the surgically implanting into the subject.

174. The method of any one of claims 170-173, wherein the acidic solution comprises a second drug.

175. The method of claim 174, wherein the second drug is an antimicrobial agent, an anti-inflammatory agent, an anti-scarring agent, a hemostatic agent, an anti-neoplastic agent, a calcium channel blocker, a leukotriene inhibitor, an antifibrotic agent, a fibrotic agent, an anesthetic, an analgesic, or a thrombolytic agent.

176. A method of producing the biodegradable covering of any one of claims 1-123, comprising:
(i) casting or melting the highly plasticized gelatin consisting essentially of gelatin and from about 35% to about 60% plasticizer to produce a film,
(ii) subjecting the film to cryoprocessing or freeze-drying, and
(iii) contacting the contacting with an aqueous solution comprising greater than 50% water and a drug, thereby coating or impregnating the film with the drug.

177. The method of claim 176, wherein the cryoprocessing or freeze-drying comprises cooling the temperature to from about 0° C. to about −40° C.

178. The method of claim 177, wherein the cryoprocessing or freeze-drying comprises cooling the temperature to from about −10° C. to about −25° C.

179. The method of claim 178, wherein the cryoprocessing or freeze-drying comprises cooling the temperature to from about −15° C. to about −20° C.

180. The method of any one of claims 176-179, wherein the cryoprocessing comprises dry air convection or applying dry air to the covering.

181. The method of claim 180, wherein the cryoprocessing or freeze-drying occurs for from about 1 hour to about 2 weeks.

182. The method of claim 181, wherein the cryoprocessing occurs for about 1-24 hours.

183. The method of any one of claims 176-179, wherein the freeze drying comprises applying reduced atmospheric pressure to the covering.

184. The method of claim 183, wherein the freeze-drying occurs for from about 1 hour to about 2 weeks.

185. The method of claim 184, wherein the freeze-drying occurs for about 1-24 hours.

186. The method of claim 183, wherein the reduced atmospheric pressure is produced via a vacuum pump.

187. The method of any one of claims 176-186, wherein the method comprises using water droplet interfacial contact angles of 60-130 degrees.

188. The method of claim 187, wherein the method comprises using water droplet interfacial contact angles of 75-90 degrees.

189. The method of any one of claims 176-188, wherein the biodegradable covering is applied to a removeable backing.

190. The method of claim 189, wherein the removeable backing comprises a silicone coating, polytetrafluoroethylene (PTFE), a plastic, a coated plastic, parylene, or graphene.

191. The method of any one of claims 176-190, wherein the covering is sterilized with radiation.

192. The method of claim 191, wherein the radiation is electron beam radiation, beta radiation, or gamma radiation.

193. The method of claim 192, wherein the radiation is applied to the covering while the covering is maintained at a cryogenic temperature.

194. The method of claim 193, wherein the radiation is applied to the covering while the covering is near or in contact with ice or dry ice.

195. The method of any one of claims 176-194, wherein the aqueous solution has a different pH than the film.

196. The method of claim 195, wherein the aqueous solution alters the pH of the film resulting in a pH of about 6-8 in the film.

197. The method of claim 196, wherein the aqueous solution alters the pH of the film resulting in a pH of about 7-7.4 in the film.

198. The method of any one of claim 195, wherein the aqueous solution has a pH of about 2-3, less than about 4, about 1-4, about 1-3, less than about 3, less than about 2.5, or less than about 2, about 1-4, about 4-7, or about 8-12.

199. The method of claim 198, wherein the aqueous solution has a pH of about 1-3, about 2-3, about 1-2.5, or about 2.5-2.8.

200. The method of any one of claims 198-199, wherein the drug is rifampin or minocycline.

201. The method of any one of claims 198-200, wherein the aqueous solution comprises rifampin and a tetracycline.

202. The method of claim 201, wherein the tetracycline is minocycline.

203. The method of any one of claims 176-197, wherein the aqueous solution has a pH of about 8-12.

204. The method of any one of claims 198-203, wherein the aqueous solution comprises an antimicrobial agent, an anti-inflammatory agent, an anti-scarring agent, a hemostatic agent, an anti-neoplastic agent, a calcium channel blocker, a leukotriene inhibitor, an antifibrotic agent, a fibrotic agent, an anesthetic, an analgesic, or a thrombolytic agent.

205. The method of any one of claims 176-204, wherein after step (iii) the pH of the film is contacted with a second aqueous solution to result in a pH in the film of about 4-6 or about 4-7.

206. The method of any one of claims 176-204, wherein the method further comprises:
(iv) subjecting the film to cryoprocessing or freeze-drying after step (iii); and
(v) contacting the film with a second aqueous solution.

207. The method of claim 206, wherein the second aqueous solution results in a pH of about 4-6 or 4-7 in the film.

208. The method of claim 207, wherein the second aqueous solution is deionized water.
